(19) United States
(12) Patent Application Publication
(10) Pub. No.: US 2024/0287462 A1
(43) Pub. Date: Aug. 29, 2024

(54) Use of Toll-Like Receptor 4 Agonists to Treat Inflammation and Tissue Injury

(71) Applicant: Wisconsin Alumni Research Foundation, Madison, WI (US)

(72) Inventors: Peiman HEMATTI, Madison, WI (US); John KINK, Madison, WI (US); Christian CAPITINI, Madison, WI (US); Matthew FORSBERG, Stoughton, WI (US); Nicholas HESS, Cottage Grove, WI (US); Zachary ROSENKRANS, Madison, WI (US); Reinier Hernandez, Madison, WI (US)

(21) Appl. No.: 18/624,848

(22) Filed: Apr. 2, 2024

Related U.S. Application Data
(63) Continuation of application No. 17/508,855, filed on Oct. 22, 2021, now abandoned.
(60) Provisional application No. 63/104,087, filed on Oct. 22, 2020.

(51) Int. Cl. C12N 5/0775; A61K 35/15; A61K 35/35; C12N 5/0786

(52) U.S. Cl. CPC C12N 5/0668; A61K 35/15; A61K 35/35; C12N 5/0645; C12N 2500/84; C12N 2502/1323; C12N 2502/1358

(57) ABSTRACT

The disclosure relates to populations of educated macrophages and monocytes generated ex vivo or in vivo, and methods of making and using the same using lipid A aminoalkyl glucosaminide phosphate molecules, such as CRX molecules or extracellular vesicles (EVs) from mesenchymal stromal cells (MSC) stimulated with CRX molecules. Also described are EVs and methods for making and using the same from MSCs exposed to CRX.

### CROSS-REFERENCE TO RELATED APPLICATIONS

This application is a continuation of U.S. patent application Ser. No. 17/508,855 filed on Oct. 22, 2021, which claims priority to U.S. Provisional Patent Application No. 63/104,087, filed Oct. 22, 2020, the contents of both are hereby incorporated by reference in its entirety.

### STATEMENT REGARDING FEDERALLY SPONSORED RESEARCH

This invention was made with government support under HL153721, CA014520 and TR000427 awarded by the National Institutes of Health. The government has certain rights in the invention.

### BACKGROUND

### Field

The disclosure relates to populations of educated macrophages and monocytes generated ex vivo or in vivo, and methods of making and using the same using lipid A aminoalkyl glucosaminide phosphate (AGP) molecules, such as CRX molecules or extracellular vesicles (EVs) from mesenchymal stromal cells (MSCs) stimulated with CRX molecules. Also described are EVs and methods for making and using the same from MSCs exposed to lipid A AGP molecules.

### Description of the Related Art

Radiation, delivered therapeutically, accidentally, or maliciously, can lead to an acute radiation syndrome (ARS) with life threatening toxicities. High-dose radiation causes inflammation and damage to highly proliferative cells such as those found in the bone marrow, GI-tract, and skin. Current treatment standard of care involves supporting victims with antibiotics and transfusions until they can undergo an allogeneic bone marrow transplant (BMT) from a suitable donor. Unfortunately, the entire BMT process can often take weeks to identify and collect cells from a donor and is difficult to perform on a large-scale in the event of widespread community exposure. Moreover, allogeneic BMTs have their own set of complications, which can include engraftment failure, opportunistic infections and graft-versus-host-disease (GVHD), making them potentially as toxic as the radiation injury itself. Consequently, adjunct therapies have received special attention for treatment of radiation injury, but the only approved treatment to date is administration of colony-stimulating factors such as G-CSF. Unfortunately, for these factors to be effective, the patient's own hematopoietic stem cells have to be spared from deleterious effects of ARS.

Attractive alternatives to treat ARS focus on allogeneic, “off-the-shelf’ cell-based therapies that can accelerate repair of tissue injury after radiation without relying on the patient's own remaining healthy cells. Ideally, cell-based therapies can be cryopreserved and ready for quick infusion without extensive tissue matching to the recipient. Among stromal cells currently explored are multipotent mesenchymal stromal cells (MSC). MSCs are capable of self-renewal and differentiation into osteocytes, chondrocytes and adipocytes, making them attractive candidates to treat tissue injury. MSCs have strong immunosuppressive properties and can control inflammation by modifying proliferation and cytokine production of immune cells. Fibroblasts are also useful to treat tissue injury and are very similar to MSCs in many properties such as morphology, surface marker profile and ability to differentiate into other tissues. MSCs have shown promise in preclinical studies in rodent models of radiation injury and have spurred human clinical trials for treating autoimmune and degenerative diseases. However, while therapeutic MSCs show promise, they often fail to demonstrate clear efficacy in many clinical trials, and have not yet been approved to treat ARS.

Many other diseases and injuries share the inflammatory and tissue damage clinical indicators of ARS and, therefore, could benefit from similar treatments with MSCs or MSC-derivatives with immunomodulatory properties. For example, MSCs are being explored as a treatment option for steroid-refractory GVHD, acute kidney injury (AKI), ankylosing spondylitis, atopic dermatitis, bronchopulmonary dysplasia, pulmonary emphysema, non-ischemic cardiomyopathy, liver allograft rejection, cirrhosis, juvenile idiopathic arthritis, type 1 and type 2 diabetes, rheumatoid arthritis, multiple sclerosis, systemic lupus erythematous, osteoarthritis, and infection, among other applications. (Forsberg et al., 2020, “Mesenchymal Stromal Cells and Exosomes: Progress and Challenges,” Frontiers in Cell and Developmental Biology, 8:665).

While studies indicate that MSCs have the potential to promote tissue repair based on their differentiation potential, the lack of MSC engraftment and differentiation at the site of injury suggest that MSCs achieve their in vivo therapeutic effects by communicating with other resident cells. Cells of monocyte and macrophage lineages (Davies et al., 2017, Biol Blood Marrow Transpl 23, 861-2) have emerged as key mediators of the biologic effects of MSCs. Macrophages can polarize generally into two broad phenotypes: classically activated (M1) macrophages, which mediate tissue damage and are considered “pro-inflammatory,” or alternatively activated (M2) macrophages, which contribute to wound healing and tissue repair and are “anti-inflammatory.” Direct co-culture of MSCs with macrophages educates the macrophages to become MSC-educated macrophages (MEMs) that increase the expression of specific surface markers (CD206) and intracellular cytokines (IL-6 and IL-10). The therapeutic utility of macrophage education by MSCs was demonstrated by enhanced survival from lethal radiation injury using a xenogeneic ARS mouse model treated with MEMs as compared to infusions of MSCs or macrophages alone (Bouchlaka et al., 2017, Biol Blood Marrow Transpl, 23(6):897-905).

While advancements have been made in treating ARS and other diseases using MSCs and MEMs, a need exists in the art for further development of new treatment methods and compositions utilizing both MSCs, or derivatives thereof such as EVs, and alternatively activated educated macrophages or exosomes.

### BRIEF SUMMARY OF THE DISCLOSURE

In one aspect, the disclosure provides a method for generating an educated CD14⁺ cell, the method comprising the steps of co-culturing (or treating) a CD14⁺ cell, which can be a macrophage or a monocyte, in vitro (i) with an EV isolated from a MSC previously exposed to a synthetic lipid A aminoalkyl glucosaminide phosphate (AGP) molecule or (ii) with a synthetic lipid A AGP molecule, until the CD14⁺ cell acquires an anti-inflammatory macrophage or monocyte phenotype. CD14⁺ cells and EVs or the CRX molecules can be co-cultured for at least 2 hours. EVs co-cultured with CD14⁺ cells can be obtained from a MSC exposed to a synthetic lipid A AGP molecule (e.g., CRX) for at least 2 hours prior to isolation of the EV.

In some embodiments, the synthetic lipid A AGP molecule is a lipid A mimetic in which the reducing sugar of lipid A has been replaced with an N-acylated aminoalkyl aglycon unit containing an 1-serine-based aglycon unit and three (R)-3-n-alkanoyloxytetradecanoyl residues comprised of varied even-numbered normal fatty acyl chains between 6 and 14 carbon atoms in length. The synthetic lipid A AGP molecule can be a CRX molecule. In some embodiments, the CRX molecule comprises three secondary acyl chains of different lengths or comprises three secondary acyl chains each having 10 carbons in length. The CRX molecule can be CRX-527.

The MSC can be exposed to about 20 ng/ml to about 2000 ng/ml CRX. The EV can be characterized as having detectable cell surface markers for CD44, CD29, CD146, CD63, CD81, CD9, and SSEA-4. The CD14⁺ cell can be co-cultured with about 20 ng/ml to about 2000 ng/mL of a CRX molecule. The MSC can also be referred to as a mesenchymal stem cell or can be a fibroblast.

In another aspect, the disclosure provides a population of CD14⁺ anti-inflammatory monocytes or macrophages produced by the methods provided herein. The anti-inflammatory monocyte or macrophage phenotype can be characterized by high expression levels of cell surface markers HLA-DR, PD-L1, CD73, and secreted proteins IL-13, G-CSF, GM-CSF, CCL-5, MIP-1a, and MIP-1b and low expression levels of cell surface markers CD16, and CD86, compared to control CD14⁺ cells.

In another aspect, the disclosure provides a method for treating an inflammatory disease or injury in a subject in need thereof comprising administering to the subject a therapeutically effective amount of an educated CD14⁺ cell population as provided herein. In accord with these methods, the subject can be administered the educated cells at an amount between about 1×10⁵ cells/kilogram and about 10×10⁹ cells/kilogram of body weight. The treated inflammatory disease or injury can be acute radiation injury, acute radiation syndrome, GVHD, AKI, Coronavirus Disease 2019 (COVID-19), or other inflammatory diseases or injuries. In some embodiments, the ARS is from a whole body radiation procedure (e.g., a whole body radiation procedure that can occur before or after a bone marrow transplant).

In a yet further aspect, the disclosure provides a method for generating an EV using a CRX molecule, the method comprising co-culturing a MSC with a CRX molecule for at least 2 hours, and isolating EVs from the co-culture, wherein an EV having a characteristic anti-inflammatory, pro-reparative activity profile is formed. The MSC can be co-cultured with about 20 ng/ml to about 2000 ng/ml CRX. The CRX molecule can be CRX-527.

In a still further aspect, the disclosure provides a population of EVs produced by the aforementioned methods for generating EVs, wherein the EVs are characterized by high expression levels of CD44, CD29, and CD146 as compared to a control EV isolated from a MSC not exposed to a CRX molecule. The EVs can include exosomes having a diameter between about 50 nm and about 200 nm and microvesicles having a diameter of an average or mean between about 300 nm to about 1100 nm.

In another aspect, the disclosure provides a method for treating an inflammatory disease or injury in a subject in need thereof, the method comprising directly administering to the subject a therapeutically effective amount of an aforementioned EV population to achieve in vivo education of effector cells. The inflammatory disease or injury can be, for example, ARS, acute radiation injury, Coronavirus Disease 2019 (COVID-19), GVHD, AKI, as well as other inflammatory diseases or injuries.

In yet another aspect, the disclosure provides a method for treating ARS in a subject in need thereof, the method comprising: administering to the subject a therapeutically effective amount of (i) a CD14⁺ cell that has been co-cultured with an EV isolated from a MSC previously exposed to a CRX molecule, generating CRX-EEMs or CRX-EEMos; (ii) an EV isolated from a MSC previously exposed to a CRX molecule, generating CRX-EVs; or (iii) a CD14⁺ cell treated with a CRX molecule (CRX-EMs or CRX-EMos), wherein ARS is effectively treated in the subject. In some embodiments, the subject has been exposed to a high dose of ionizing radiation.

Effective treatment of ARS can be indicated by an increase in cell types (e.g., white blood cells) when performing a complete blood count (CBCs) of the subject as compared CBCs of the subject in the days following an exposure to radiation. Effective treatment can also be indicated by the subject having reduced symptoms of acute radiation syndrome. In some embodiments, the subject treated for ARS is immune-competent.

In yet another aspect, the disclosure provides a method for treating or preventing GVHD in a subject undergoing a transplant (e.g., a bone marrow transplant), the method comprising administering to the subject a therapeutically effective amount of (i) a CD14⁺ cell that has been co-cultured with an EV isolated from a MSC previously exposed to a CRX molecule, generating CRX-EEMs or CRX-EEMos; (ii) an EV isolated from a MSC previously exposed to a CRX molecule, generating CRX-EVs; or (iii) a CD14⁺ cell treated with a CRX molecule (CRX-EMs or CRX-EMos), wherein the GVHD is suppressed in the subject. The EV can be administered to the subject in multiple doses and in an interval of about every two days to about every seven days.

In yet another aspect, the disclosure provides a method for treating or preventing acute kidney injury in a subject, the method comprising: administering to the subject a therapeutically effective amount of (i) a CD14⁺ cell that has been co-cultured with an EV isolated from a MSC previously exposed to a CRX molecule, generating CRX-EEMs or CRX-EEMos; (ii) an EV isolated from a MSC previously exposed to a CRX molecule, generating CRX-EVs; or (iii) a CD14⁺ cell treated with a CRX molecule (CRX-EMs or CRX-EMos), wherein acute kidney injury is effectively treated in the subject. The acute kidney injury can be caused by rhabdomyolysis, chemotherapy, or other processes involving oxidative damage to kidneys.

### INCORPORATION BY REFERENCE

All publications, patents, and patent applications mentioned in this specification are herein incorporated by reference to the same extent as if each individual publication, patent, and patent application was specifically and individually indicated to be incorporated by reference.

### BRIEF DESCRIPTION OF DRAWINGS

The disclosure will be better understood and features, aspects, and advantages other than those set forth above will become apparent when consideration is given to the following detailed description thereof. Such detailed description refers to the following drawings.

FIG. 1 is a graph that illustrates the use of flow cytometry to compare the mean fluorescent intensity of cell surface markers of EVs derived from human MSCs treated with toll-like receptor 4 (TLR4) agonist CRX-527 (wherein the agent is abbreviated as “CRX,” and the EVs as “CRX-EV” herein) compared to EVs from unstimulated MSCs (MSC-EV).

FIG. 2 is a graph that shows statistically significant differences in median fluorescent intensity (MFI) of exosome surface markers between CRX-EVs versus MSC-EVs. P values: CD44<0.000001, CD29=0.000004, CD146=0.000012, and SSEA-4=0.01.

FIG. 3 are graphs that illustrate the use of flow cytometry to examine the percentage of human monocyte subsets observed between monocytes treated with EVs from unstimulated MSCs (EEMos), monocytes treated with EVs from MSCs stimulated with the TLR4 agonist lipopolysaccharide (LPS-EEMos) or MSCs stimulated with CRX (CRX-EEMos), and monocytes treated with EVs from MSCs stimulated with CRX for a short “pulse” period (CRX-pulse-EEMos). Statistical values *P≤0.05, **P≤0.005, ***P≤0.0005, ****P≤0.0001 between groups is designated by bars. Descriptions and full names of the recited cell types are provided elsewhere herein.

FIG. 4A is a graph that shows that human CRX-EEMos significantly enhance survival against lethal radiation in an ARS mouse model as compared to mice treated with phosphate-buffered saline (PBS) (P=0.001) or with human monocytes treated with EVs from unstimulated MSCs (EEMos) (P=0.02).

FIG. 4B is a graph that shows that human CRX-EEMos reduce the clinical side effects of inflammation and tissue damage associated with lethal ARS in mice (days 22 to 48) as compared to mice treated with PBS or with human monocytes treated with EVs from unstimulated MSCs (EEMos) (P=0.04).

FIG. 4C is a graph that shows that human 2-hour CRX-pulse-EEMos are not protective against lethal radiation when compared to mice treated with PBS in an ARS mouse model.

FIG. 5A is a graph that shows that direct treatment of mice with EVs obtained from human MSCs stimulated with CRX (CRX-EV) significantly enhances survival (P=0.0038) against lethal radiation as compared to mice treated with PBS in an ARS mouse model.

FIG. 5B is a graph that shows that direct treatment with EVs obtained from human MSCs stimulated with LPS (LPS-EV) significantly enhance survival (P=0.003) against lethal radiation in an ARS mouse model as compared to mice treated with PBS (P=0.004) or with EVs from unstimulated MSCs (MSC-EV).

FIG. 5C is a graph that shows that EVs obtained from human MSCs treated with a 2-hour pulse of CRX (CRX-pulse-EV) enhanced survival (P=0.1) against lethal radiation in an ARS mouse model as compared to mice treated with PBS. The median survival in the mice increased from 10 days when treating with PBS to 32 days treating with CRX-pulse-EV.

FIG. 6A is a graph showing percent survival in immunocompetent ICR mice during the days post radiation in mice treated with PBS, EVs from unstimulated MSCs (MSC-EV), or EVs from MSCs stimulated with CRX (CRX-EV).

FIG. 6B is a graph showing the percent weight change of immunocompetent ICR mice during the days post radiation of mice treated with PBS, EVs from unstimulated MSCs (MSC-EV), or CRX-EV.

FIG. 6C is a graph showing the mean clinical score in immunocompetent ICR mice during the days post radiation in mice treated with PBS, EVs from unstimulated MSCs (MSC-EV), or EVs from MSCs stimulated with CRX (CRX-EV).

FIG. 7A is a graph that shows that treatment with human monocytes directly exposed ex vivo to CRX (CRX-EMos) or human monocytes treated with EVs from MSC stimulated with CRX (CRX-EEMos) significantly enhance survival (P=0.0037 and P=0.0011, respectively) against lethal radiation in an ARS mouse model as compared to mice treated with PBS or mice treated with EVs from unstimulated MSCs (EEMos) (P=0.03 and P=0.02, respectively).

FIG. 7B is a graph that shows that treatment with human CRX-EMos or with CRX-EEMos improve clinical side effects from lethal radiation in an ARS mouse model as compared to mice treated with PBS or EEMos. The effect was significant at day 20 to day 40 using either CRX-EMos (P=0.01) or CRX-EEMos (P=0.04) when compared to PBS-treated mice. This effect was also significant at day 40 to day 60 using CRX-EEMos when compared to the EEMos (P=0.03).

FIG. 8 is a survival graph of mice transplanted with human peripheral blood mononuclear cells (PB-MNC) alone, treated with exosomes from CRX stimulated MSCs (CRX-EV) at 0, 1, 3, and 5 days post-transplant, or CRX-EV at 0, 7, 14, and 21 days post-transplant.

FIG. 9A is a graph showing the number of human T cells in the liver of mice treated with PB-MNC, CRX-EV at 0, 1, 3, and 5 days post-transplant, and CRX-EV at 0, 7, 14, and 21 days post-transplant.

FIG. 9B is a graph showing the percentage of a pathogenic CD4⁺/CD8⁺ double positive T cell (DPT) population in mice treated with PB-MNC, CRX-EV at 0, 1, 3, and 5 days post-transplant, or CRX-EV at 0, 7, 14, and 21 days post-transplant.

FIG. 10 is a graph showing the concertation of IFNγ produced by unstimulated and stimulated human T cells with and without the addition of CRX-EV in vitro.

FIG. 11 is a survival graph of mice transplanted with isolated human T cells alone, treated with CRX-EV at days 0, 1, 3, and 5 post-transplant, or CRX-EV at days 0, 7, 14, and 21 post-transplant.

FIG. 12A is a schematic showing the rhabdomyolysis-induced AKI (RM-AKI) mouse model protocol.

FIG. 12B is a schematic showing the cisplatin-induced AKI (CP-AKI) mouse model protocol.

FIG. 13A shows PET imaging of the biodistribution of radiolabeled CRX-EVs in the RM-AKI model mice and saline control mice.

FIG. 13B shows PET imaging of the biodistribution of radiolabeled CRX-EVs in the CP-AKI model mice and healthy controls. K stands for kidney and Bl stands for bladder.

FIG. 14A is a graph showing the percent survival in mice treated with CRX-EVs and control PBS in the 8 mL/kg glycerol group of the RM-AKI model.

FIG. 14B is a graph showing the percent survival in mice treated with CRX-EVs and control PBS in the 7 mL/kg glycerol group of the RM-AKI model.

### DETAILED DESCRIPTION OF THE DISCLOSURE

For the purposes of promoting an understanding of the principles of the disclosure, reference will now be made to embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

### Definitions

As used in the specification, articles “a” and “an” are used herein to refer to one or to more than one (i.e., at least one) of the grammatical object of the article. By way of example, “an element” means at least one element and can include more than one element.

“About” is used to provide flexibility to a numerical range endpoint by providing that a given value can be “slightly above” or “slightly below” the endpoint without affecting the desired result. The term “about” in association with a numerical value means that the numerical value can vary by plus or minus 5% or less of the numerical value.

Throughout this specification, unless the context requires otherwise, the word “comprise” and “include” and variations (e.g., “comprises,” “comprising,” “includes,” “including”) will be understood to imply the inclusion of a stated component, feature, element, or step or group of components, features, elements or steps but not the exclusion of any other integer or step or group of integers or steps.

As used herein, “and/or” refers to and encompasses any and all possible combinations of one or more of the associated listed items, as well as the lack of combinations where interpreted in the alternative (“or”).

Recitation of ranges of values herein are merely intended to serve as a succinct method of referring individually to each separate value falling within the range, unless otherwise indicated herein. Furthermore, each separate value is incorporated into the specification as if it were individually recited herein. For example, if a range is stated as 1 to 50, it is intended that values such as 2 to 4, 10 to 30, or 1 to 3, etc., are expressly enumerated in this disclosure. These are only examples of what is specifically intended, and all possible combinations of numerical values between and including the lowest value and the highest value enumerated are to be considered to be expressly stated in this disclosure.

Unless otherwise defined, all technical terms used herein have the same meaning as commonly understood by a person of ordinary skill in the art to which this disclosure belongs.

### Educated CD14⁺ Cells

The present disclosure broadly relates in part to alternatively activated CD14⁺ cells (macrophages or monocytes) as well as methods for making and using such cells to treat inflammation or tissue injury. The educated CD14⁺ cell is advantageously a cell with an anti-inflammatory, immunosuppressive, tissue protective and tissue reparative phenotype. Methods of this disclosure broadly relate to deriving extracellular vesicles (EVs, which can include exosomes and other extracellular microvesicles) from MSCs treated with a synthetic TLR4 agonist, such as a synthetic lipid A aminoalkyl glucosamine phosphate, which can be a CRX molecule, as well as to the EV obtained. Reference to a CRX molecule (or CRX) in connection with various embodiments herein refers to compound CRX-527, an exemplary but non-limiting example of such compounds, with the understanding that other CRX molecules or other TLR4 agonists are also suitably employed and are within the scope of reference to a CRX molecule. EV so obtained from MSCs are referred to as CRX-EV. The disclosure also relates to exposing CRX-EV to cultured CD14⁺ cells (monocytes (Mo) and macrophages (M)) to generate CRX-EV-educated monocytes (CRX-EEMos) and CRX-EV-educated macrophages (CRX-EEMs). Still further, and in contrast to methods wherein EVs from MSC treated with CRX educate CD14⁺ cells, the disclosure also relates to methods for obtaining CRX-educated monocytes (CRX-Mos) and macrophages (CRX-Ms) by directly exposing cultured CD14⁺ cells to CRX. The disclosure also broadly relates to methods for treating human and non-human subjects using CRX, CRX-EVs, and educated cells obtained in the referenced methods, including, CRX-EEMs, CRX-EEMos, CRX-Mos, and CRX-Ms to treat or reduce effects of inflammatory and tissue injuries, including but not limited to those discussed, infra.

In one aspect of the disclosure, MSCs are cultured in the presence of a CRX molecule to generate CRX-primed MSCs. CRX-EV isolated from the CRX-primed MSCs are co-cultured with CD14⁺ monocytes or macrophages to yield educated macrophages (CRX-EEMs) or educated monocytes (CRX-EEMos) with a characteristic cytokine profile, expression profile, and phenotype as described herein. CRX-EVs, educated macrophages, or educated monocytes generated by the methods of the present disclosure can be used to treat or prevent a disease by administering the EVs or educated cells to a subject in need thereof.

As used herein, “educated” cells refer to anti-inflammatory, immunosuppressive, tissue protective, or tissue reparative, monocytes and macrophages generated ex vivo by co-culturing a CD14′ monocyte or macrophage with CRX-EV. “Educated” cells can also be anti-inflammatory, immunosuppressive, tissue protective, or tissue reparative, monocytes and macrophages generated ex vivo by exposing CD14⁺ monocytes or macrophages directly to a CRX molecule. “Educated” cells can also be cells generated in vivo by treating a subject directly with CRX-EV or a CRX molecule. Like ex vivo-generated educated monocytes and macrophages, these in vivo-educated cells are also believed to be anti-inflammatory, immunosuppressive, tissue protective, or tissue reparative monocytes or macrophages. Similarly, “educated” cells can also be cells generated in vivo by treating a subject directly with LPS-EV, wherein such in vivo-educated cells are believed to be anti-inflammatory, immunosuppressive, tissue protective, or tissue reparative monocytes or macrophages.

The anti-inflammatory monocyte or macrophage phenotype can be characterized as HLA-DR high, PD-L1 high, CD73 high, CD16 low, CD163 low, and CD86 low, compared to control CD14⁺ cells.

### Co-Culture

Provided herein are methods for producing CRX-EEMs and CRX-EEMos. CD14⁺ cells are cultured with CRX-EVs to yield CRX-EEMs or CRX-EEMos, respectively. Also provided are methods for producing educated macrophages (CRX-Ms) and monocytes (CRX-Mos) by directly exposing the CD14⁺ cells to a CRX molecule. Methods of treating CD14⁺ cells with MSCs or with EVs to generate MSC-educated macrophages (MEM) or exosome-educated macrophages (EEMs), respectively, have been described, see U.S. Pat. No. 8,647,678 and U.S. Patent Publication No. 2016/0082042, each of which is incorporated herein by reference as if set forth in its entirety. Methods of co-culturing CD14⁺ cells with EVs from lipopolysaccharide-treated MSC to generate LPS-exosome-educated macrophages (LPS-EEM) and LPS-exosome-educated monocytes (LPS-EEMos) have been described previously; see U.S. Patent Publication No. 2019/0249144, which is incorporated herein by reference in its entirety.

CD14⁺ cells are co-cultured ex vivo with a TLR4 agonist (e.g., CRX) or EV's produced from a TLR4 agonist exposed to MSC (e.g., CRX-EVs) in any culture medium, with or without added growth factors, cytokines or serum of different origins that are known in the art suitable for survival and growth of the co-culture components. CD14⁺ cells can be co-cultured in culture plates, culture flasks, culture bags, or in hollow fiber systems. To generate educated monocytes, the co-cultures can be maintained for between at least 30 minutes and 5 days. Co-cultures can generate educated monocytes with the desired immunophenotype after 30 minutes, 1 hour, 2 hours, 3 hours, 4 hours, 5 hours, 8 hours, 10 hours, 12 hours, 15 hours, 18 hours, 20 hours, 22 hours, 24 hours, 36 hours, 48 hours, 50 hours, 72 hours, 1 day, 2 days, 3 days, 4 days, or 5 days. In some embodiments, co-cultures yield educated monocytes after 30 minutes, 3 hours, after 18 hours, or after 24 hours. In some embodiments, co-cultures yield educated monocytes after 48 hours.

To educate macrophages, monocytes need to be differentiated into macrophages. In general, to produce a population of educated macrophages starting with a population of CD14⁺ monocytes, CD14⁺ cells are cultured for at least 3 days to generate macrophages to later be educated. The resulting CD14⁺ cells differentiated to macrophages can be co-cultured with a CRX molecule or CRX-EVs for at least 2 hours (e.g., 2 hours, 10 hours, 14 hours, 18 hours, or 24 hours) to generate educated macrophages. In some embodiments, CD14⁺ cells are cultured for about 5 days to 10 days (e.g., 5 days, 6, days, 7 days, 8 days, 9 days, or 10 days) to generate macrophages to later be educated. In some embodiments, the resulting CD14⁺ cells differentiated to macrophages can be co-cultured with a CRX molecule or CRX-EVs for less than 2 hours to generate educated macrophages. A CRX molecule or CRX-EVs can be added in a single dose or repeated doses to CD14⁺ cultures to generate educated CD14⁺ cells.

To generate educated macrophages, the co-cultures can be maintained for between 1 and 20 days. Co-cultures can generate educated macrophages with the desired immunophenotype after 30 minutes, 1 hour, 2 hours, 3 hours, 4 hours, 5 hours, 6 hours, 7 hours, 8 hours, 9 hours, 10 hours, 12 hours, 14 hours, 16 hours, 18 hours, 20 hours, 22 hours, 24 hours, 48 hours, 1 day, 2 days, 3 day, 4 days, 5 days, 6 days, 7 days, 8 days, 9 days, 10 day, 11 days, 12 days, 13 day, 14 days, or more than 15 days. In some embodiments, co-cultures yield educated macrophages after 10 days. In some embodiments, co-cultures yield educated macrophages after 3 days. In one embodiment, co-cultures yield educated macrophages after 1 day. In one embodiment, co-cultures yield educated macrophages in 2 hours or less.

In some cases, CRX-EVs are subjected to additional purification steps prior to use in co-culture. In one embodiment, CRX-EVs are purified using centrifugation, wherein smaller exosomes are separated from larger microvesicles and other EV by including a lower speed (10-20,000×g) centrifugation step before a final 100,000×g centrifugation step to isolate the exosomes.

For co-cultures of the present disclosure, monocytes or macrophages can be co-cultured with CRX-EV wherein cells are in direct physical contact with the EV. Additionally, monocytes or macrophages can be co-cultured with condition media that contains EVs (i.e., with EVs that have not gone through the purification process). Alternatively, the co-culture components can be placed in sub-compartments that are in fluid communication but separated by a semi-permeable membrane. The semi-permeable membrane allows the exchange of soluble medium components and factors secreted by the CRX-primed cells but not the cells themselves. In these embodiments, the pores within the semi-permeable membrane are sufficiently small to prevent cell penetration but large enough to allow soluble medium components to pass across the membrane, and are typically are between 0.1 μm and 1 μm, but other pore sizes can be suitable.

Various cell separation and isolation methods are known in the art and can be used to separate the educated macrophages or educated monocytes depending on factors such as the desired purity of the isolated cell populations. For example, macrophages are strongly adherent to solid culture surfaces and monocytes are weakly adherent to culture surfaces which can aid in separating and isolating the educated CD14⁺ cells. Educated macrophages or educated monocytes can be isolated using flow cytometry, magnetic-based sorting, being scraped from plates, a digestive process (e.g., trypsinization and EDTA), or low speed centrifugation. In some embodiments, educated macrophages or educated monocytes can be separated from CRX-EVs by removal of the culture medium containing the CRX-EVs followed by multiple washing steps. Educated macrophages can be maintained in culture in any medium that supports macrophages in vitro. Educated monocytes can be maintained in culture in any medium that supports monocytes in vitro. Also, educated macrophages or educated monocytes can be stored using methods known in the art including, but not limited to, refrigeration, cryopreservation, vitrification, and immortalization.

As used herein, “CD14⁺ cell” refers to a monocyte or a macrophage. CD14⁺ cells can be derived from any suitable source such as from blood, mobilized blood, bone marrow, embryonic stem cells (ESCs), induced pluripotent stem cells (iPSCs), or other tissues. CD14⁺ used in the methods and compositions described herein can be human CD14⁺ cells. The skilled artisan will appreciate the advantageous efficiency of generating macrophages from peripheral blood monocytes for co-cultures. Alternatively, macrophages can also be isolated from cellular outgrowth of a tissue sample taken from an individual or from pluripotent stem cells.

Monocytes can be cultured for various times and under various conditions before and after co-culture or can be added to the exosomes or CRX-containing medium directly for co-cultures. In one embodiment, monocytes are harvested from a subject by leukapheresis. In one embodiment, CD14⁺ cells are isolated from peripheral blood. In one embodiment, CD14⁺ cells are isolated from peripheral blood of a patient who has first been treated with an agent including but not limited to G-CSF, GM-CSF, Plerixafor or the like to mobilize cells into the peripheral blood. In one embodiment, CD14⁺ cells are isolated from peripheral blood with G-CSF stimulation. In one embodiment, CD14⁺ cells are isolated from bone marrow aspirates. In one embodiment CD14⁺ cells are isolated from tissues or organs of interest. In one embodiment CD14⁺ cells are derived from pluripotent stem cells such as embryonic stem cells or induced pluripotent stem cells.

As used herein, “macrophage” refers to a mononuclear phagocyte characterized by expression of CD14 and lack of expression of mesenchymal stromal cell markers.

As used herein, “monocytes” are white blood cells that can differentiate into macrophages when recruited to tissues and can influence both innate and adaptive immune system.

As used herein, “peripheral blood mononuclear cell” or “PB-MNC” refers to any peripheral blood cell having a round nucleus. PB-MNCs can include lymphocytes (T cells, B cells, NK cells) and monocytes.

As used herein, “high” when used to characterize expression means that the cells are characterized by higher expression of a particular cytokine, chemokine, growth factor or cell surface marker compared to control macrophages or monocytes cultured under the same conditions without CRX or CRX-EVs. Expression of markers can be measured by any means known in the art, including but not limited to, gene expression analysis (qPCR), Western blot, secretion product measurement by ELISA, multiplex detection systems, transcriptome analysis or flow-cytometry. For example, “IL-6 high” indicates that monocytes/macrophages co-cultured with a CRX molecule or CRX-EVs express higher amounts of IL-6 than monocytes/macrophages that have not been co-cultured with a CRX molecule or CRX-EVs. Similarly, “low” means that the cells are characterized by lower expression of a particular cytokine compared to control macrophages or monocytes cultured under the same conditions without a CRX molecule or CRX-EVs. For example, “IL-12 low” indicate that monocytes/macrophages co-cultured with a CRX molecule or CRX-EVs express lower amounts of IL-12 than monocytes/macrophages that have not been co-cultured with a CRX molecule or CRX-EVs. “Low” can also mean that the expression levels or secretion levels are below the detection limit.

### Primed Stromal Cells and EV

The skilled artisan will appreciate that monocytes, macrophages, MSCs, and EV employed in methods described herein can be cultured or co-cultured in any medium that supports their survival and growth. CD14⁺ cells can be co-cultured in culture plates, culture flasks, culture bags, or in hollow fiber systems. In some embodiments, the medium is a serum-free medium (SFM) supplemented with chemically defined mammalian serum supplement. In some embodiments, the medium is supplemented SFM including but not limited to X-VIVO™ 15, CTS™ STEMPRO™ MSC serum-free media (SFM), STEMPRO™-34 SFM or macrophage SFM. Conventional culture media supplemented with serum or an animal supplement depleted of endogenous EVs that can be present in the serum can also be used. EVs can be isolated from serum by means such as ultracentrifugation, ultrafiltration, size exclusion chromatography, tangential flow centrifugation, density gradient centrifugation, gel filtration, or affinity purification. EVs can be isolated from serum prior to culturing the cells or isolated along with the production of CRX-EVs. Suitable serum from which endogenous EVs can be isolated include but are not limited to fetal bovine serum, fetal calf serum, human serum, and human A/B serum. For short-term cultivation of about 1 day to about 3 days, conventional culture medium without serum has also been used. In one embodiment, the medium uses human platelet lysates to replace the human A/B serum in the culture medium for macrophage and monocyte cultures. In some embodiments, in order to isolate EVs for the cells of interest, the culture medium is free of endogenous EVs present in either mammalian serum or protein supplements derived from humans or animals such as human platelet lysate. MSCs, EV, and macrophages can be autologous, syngeneic, allogeneic, third party or ex vivo generated with respect to one another.

As used herein, the term mesenchymal stromal cell or MSC refers to fibroblast-like cells that reside within virtually all tissues of a postnatal individual. An ordinarily skilled artisan will appreciate that the cells referred to herein as MSCs are known in the art as mesenchymal stromal cells, mesenchymal stem cells, marrow stromal cells, multipotent stem cells, and other names. An MSC within the scope of this disclosure is any cell that can differentiate into osteoblasts, chondrocytes, myocytes, and adipocytes. An MSC within the scope of this disclosure is positive for the expression of CD105, CD73, and CD90 while lacking expression of CD45, CD34, CD14 or CD11b, CD79u or CD19, and HLA-DR surface molecules (see Dominici et al., 2006, Minimal criteria for defining multipotent mesenchymal stromal cells. The International Society for Cellular Therapy position statement, Cytotherapy, 8(4):315-317). While these markers are known to characterize MSCs derived from most tissues, it is understood in the art that MSCs or fibroblasts from some sources could exhibit differences in cell surface marker expression. MSCs can differentiate into cells of the mesenchymal lineage. In some embodiments, MSCs are co-cultured with CD14⁺ cells to generate MSC-educated macrophages (referred to herein as MEMs). In some embodiments, the MSCs are CRX-primed MSCs (CRX-MSC) that have been cultured in the presence of a CRX molecule.

MSCs, fibroblasts, and other cells described herein for use in the methods or compositions of the present disclosure can be derived or isolated from any suitable source. MSCs and fibroblasts can be isolated from tissues including but not limited to bone marrow, lung, cornea, intestines, testis, tendon, adipose, muscle, liver, vertebral, placental, umbilical, and amniotic tissue. In one embodiment, MSCs are isolated from bone marrow (BM-MSCs). In one embodiment, MSCs are differentiated from embryonic stem cells (ESCs) or induced pluripotent stem cells (iPSCs).

MSCs can be cultured with a TLR4 agonist to produce EVs. As used herein, CRX-MSC refers to an MSC that has been cultured in the presence of CRX for an amount of time sufficient to generate therapeutically effective EVs. The MSCs can be cultured in the presence of CRX for at least 2 hours (e.g., 3 hours, 4 hours, 5 hours, 6 hours, 7 hours, 8 hours, or 9 hours) prior to isolating the EV. In some embodiments, the MSCs can be cultured in the presence of CRX for at least 10 hours (e.g., at least 10 hours, at least 12 hours, at least 15 hours, at least 18 hours, at least 20 hours, at least 24 hours, at least 28 hours, at least 32 hours, or at least 48 hours) prior to isolating the EV in any suitable culture medium known in the art that will support the growth and survival of the MSCs. For example, the pulse method described herein produced therapeutically effective EVs when MSCs were treated with CRX for 2 hours. It is also possible to culture MSCs repeatedly in the presence of a CRX molecule, harvesting the EV-containing medium after a sufficient culture time, then replacing the medium with fresh CRX-containing medium, then repeating the harvest and culture cycle several times, for at least 72 hours. The CRX molecule is present in the culture medium at about 1 ng/ml to about 10 μg/ml. It is preferred that the CRX molecule be provided for a time sufficient to raise IL-6 expression level in treated MSCs to 5-10 fold higher than in the control MSCs. This change might also be accomplished by treating for shorter times at a somewhat higher concentration of the TLR4 agonist.

In addition to natural TLR4 agonists such as lipopolysaccharide (LPS), which can be operably employed in the disclosure, synthetic lipid A AGP molecules and the CRX class of synthetic lipid A AGP mimetics are known and described in the art. See, for example, Stover et al., 2004, Structure-activity relationship of synthetic Toll-Like Receptor 4 agonists, J. Biol. Chem., 279(6):4440-4449 and Legat et al., 2010, CD-14 independent responses induced by a synthetic lipid A mimetic, Eur. J. Immunol., 40:797-802, each of which is incorporated herein by reference in their entirety. Lipid A is the active component of LPS and is variable across Gram-negative species. In vitro studies have shown the relationship between the acyl chain lengths of lipid A mimetic structures and TLR4 activation, with chain lengths greater than 8 carbons being favorable for TLR4 stimulation.

In some embodiments, the synthetic lipid A AGP molecule is a lipid A mimetic in which the reducing sugar of lipid A has been replaced with an N-acylated aminoalkyl aglycon unit containing an 1-serine-based aglycon unit and three (R)-3-n-alkanoyloxytetradecanoyl residues (secondary acyl chains) comprised of varied even-numbered normal fatty acyl chains between 6 and 14 carbon atoms in length (e.g., 6 carbons (C), 8 carbons, 10 carbons, 12 carbons, or 14 carbons). In some embodiments, the synthetic lipid A AGP molecule is a CRX molecule. In some embodiments, the CRX molecule comprises three secondary acyl chain each having 10 carbons in length (C10, C10, C10, e.g., CRX-527). In other embodiments, the CRX molecule comprises three secondary acyl chains of different lengths (e.g., C6, C8, C10 or C10, C12, or C14). In other embodiments, the CRX molecule comprises three secondary acyl chains having two chains of the same length and one chain a different length (e.g., C6, C10, C6; or C10, C6, C10; or C12, C10, C12). In some embodiments, the synthetic lipid A AGP mimetic is a CRX molecule with secondary acyl chains of at least 8 carbons. In some embodiments, the synthetic lipid A AGP mimetic is a CRX molecule with secondary acyl chains of at least 10 carbons

In some embodiments, the synthetic lipid A AGP mimetic is CRX-527, having the structure of Formula I.

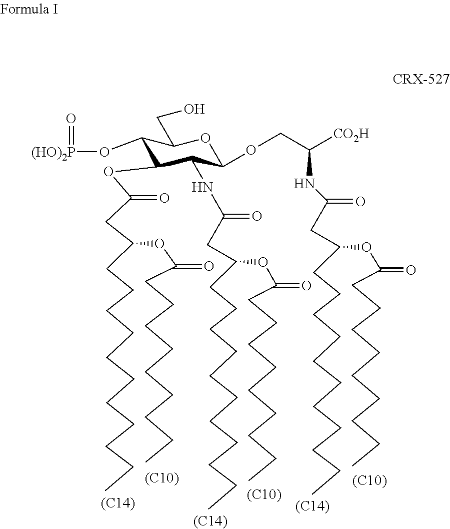

In some embodiments, the synthetic lipid A AGP mimetic can be CRX-512, CRX-524, CRX-526, CRX-527, CRX-547, CRX-555, CRX-560, CRX-565, CRX-566, CRX-567, CRX-568, CRX-569, CRX-570, CRX-601, CRX-657, or CRX-675. In some embodiments, the synthetic lipid A mimetic can be combinations of any of the foregoing.

Synthetic lipid A mimetics can be prepared by chemical synthesis by methods known in the art.

In some embodiments, more than one type of CRX molecule (e.g., 2, 3, 4, or 5 different CRX molecules) can be used in the methods described herein. For example, an MSC can be co-cultured with one or more CRX molecules (e.g., CRX-527 and CRX-555) to produce CRX-EVs.

In some embodiments, a CRX molecule can be replaced or administered to CD14⁺ cells or a subject in combination with another TLR4 agonist. In addition to the synthetic lipid A mimetics mentioned above, exemplary TLR4 agonists suitable for use in the methods described herein include but are not limited to LPS, VSV glycoprotein G, RSV fusion protein, MMTV envelope protein, mannan, glucuronoxylomannan, glycosylinositolphospholipids, HSP60, HSP70, fibrinogen, nickel, HMGB1 (from Lee et al, 2012, Nature Reviews Immunology 12, 168-172), 1Z105 (a substituted pyrimido[5,4-b]indole (Goff, P H et al., 2015, J Virol 89,6), Glucopyranosyl Lipid Adjuvant (GLA) (Arias et al., 2012, PLOS ONE 7(7): e41144), and other synthetic lipid A mimetics (e.g., aminoalkyl glucosaminide 4-phosphates, Evans et al., 2003, J Expert Review of Vaccines, 2 or E5564), GSK1795091, palmitic acid, drugs targeting TLR4 (e.g., buprenorphine, carbamazepine, ethanol, fentanyl, levorphanol, lipopoylsaccharides (LPS), methadone, morphine, oxcarbazepine, oxycodone, pethidine, morphine-3-glucuronide, or tapentadol), MPL, MPLA, RC-529, lipid A, GLA, ONO-4007, OM-174, E6020 (Fox et al., 2010, Subcell Biochem, 53:303-21) as well as TLR4 agonist peptides (Shanmugam et al., 2012, PLOS ONE 7(2): e30839).

As used herein, “extracellular vesicles” refers collectively to EVs, exosomes, microvesicles, extracellular matrix compositions, isolated extracellular matrix components and fragments or derivatives thereof, exosomes purified from an extracellular matrix, and combinations thereof. Extracellular factors are used in co-culture with CD14⁺ cells to educate macrophages or monocytes in a tissue-specific manner. As used herein, EV includes both exosomes and microvesicles, and can refer to preparations that contain more of one factor or another, whether used with or without further enrichment for one or another. Extracellular factors from CRX-treated MSCs can also be used to directly treat a subject as described herein.

As used herein, “exosomes” refer to small lipid vesicles released by a variety of cell types. Exosomes are generated by inward or reverse budding, resulting in particles that contain cytosol and exposed extracellular domains of certain membrane-associated proteins (Stoorvogel et al., 2002, Traffic 3:321-330). Methods of preparing exosomes from cells are known in the art. See, e.g., Raposo et al., 1996, J. Exp. Med. 183:1161. In one method, exosomes are recovered from conditioned culture medium by centrifugation. In one method, exosomes are directly applied by using conditioned media. Exosomes suited for use in the methods set forth herein can be derived fresh or can be previously frozen aliquots kept as a composition, thawed, and added in a single dose or repeated doses to CD14⁺ cultures to generate educated macrophages. In some embodiments, exosome preparations can also include microvesicles.

Exosomes can have, but are not limited to, a diameter of about 10 nm to about 300 nm (e.g., 10 nm, 15 nm, 20 nm, 25 nm, 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 110 nm, 120 nm, 130 nm, 140 nm, 150 nm, 160 nm, 170 nm, 180 nm, 190 nm, 200 nm, 210 nm, 220 nm, 230 nm, 240 nm, 250 nm, 260 nm, 270 nm, 280 nm, 290 nm, or 300 nm). In some embodiments, the exosomes can have, but are not limited to, a diameter between 20 nm and 250 nm, 30 nm and 200 nm, or about 50 nm and 150 nm. Exosomes can have a diameter of an average or mean between about 50 nm to about 200 nm. Exosomes can be isolated or derived from any cell type that resides in the target tissue of interest which can be isolated and cultured for a period of time appropriate for the isolation of exosomes.

Exosomes can be distinguished from microvesicles based on size. Microvesicles can have, but are not limited to, a diameter of about 300 nm to about 1100 nm (e.g., 300 nm, 350 nm, 400 nm, 450 nm, 500 nm, 550 nm, 600 nm, 650 nm, 700 nm, 750 nm, 800 nm, 850 nm, 900 nm, 1000 nm, 1050 nm, or 1100 nm). In some embodiments, microvesicles can have, but are not limited to, a diameter between 350 nm and 1050 nm, 375 nm and 1025 mn, or about 400 nm and 1000 nm. Microvesicles can be isolated or derived using methods similar to those used to isolate or derive exosomes.

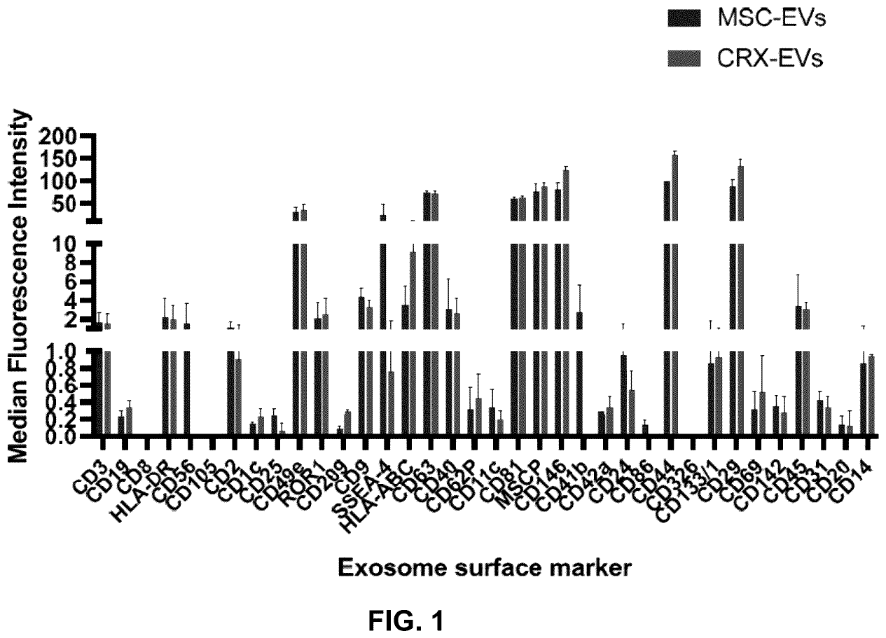

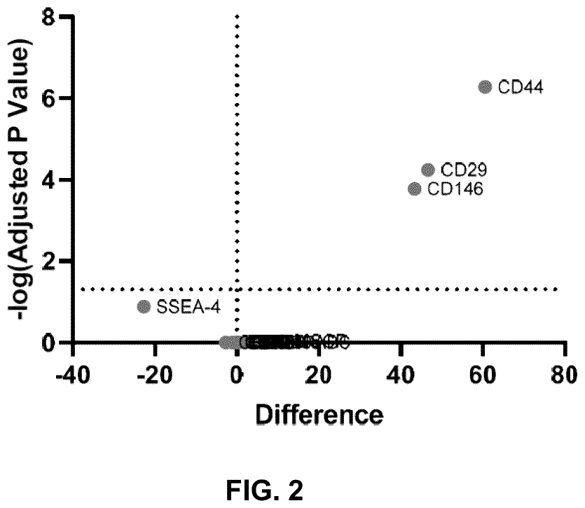

In some embodiments, exosomes and microvesicles are derived from MSCs treated with CRX (CRX-EV). In some embodiments, EVs are isolated from the CRX-primed MSC culture by harvesting medium containing the EVs. Multiple cycles of EV isolation can be performed from a single population of MSCs in culture. For example, medium harvested from MSCs after a first CRX priming can be replaced with fresh media containing a CRX molecule for another round of CRX priming and EV isolation. In some embodiments, MSCs can be primed with a CRX molecule at about 20 ng/ml to about 500 ng/ml. In some embodiments, MSCs can be primed with a CRX molecule at about 25 ng/ml, about 50 ng/ml, about 75 ng/ml, about 100 ng/ml, about 125 ng/ml, about 150 ng/ml, about 175 ng/ml, about 200 ng/ml, about 225 ng/ml, about 250 ng/ml, or about 500 ng/ml. Thirty-seven exosomal surface markers were screened by flow cytometry in MSC-EVs and CRX-EVs (FIG. 1). Three markers, CD44 (P<0.000001), CD29 (P=0.000004) and CD146 (P=0.000012) were expressed at significantly higher levels while one marker, SSEA-4 (P=0.01) at a lower level in the CRX-EVs compared to control MSC-EVs. In general, CRX-EVs are characterized by high CD44, CD29, and CD146 expression and low SSEA-4 cell surface marker expression as compared to EVs from MSCs that have not been stimulated. See FIG. 2. CRX-EVs can also be characterized by detectable surface markers for CD44, CD29, CD146, CD63, CD81, CD9, and SSEA-4.

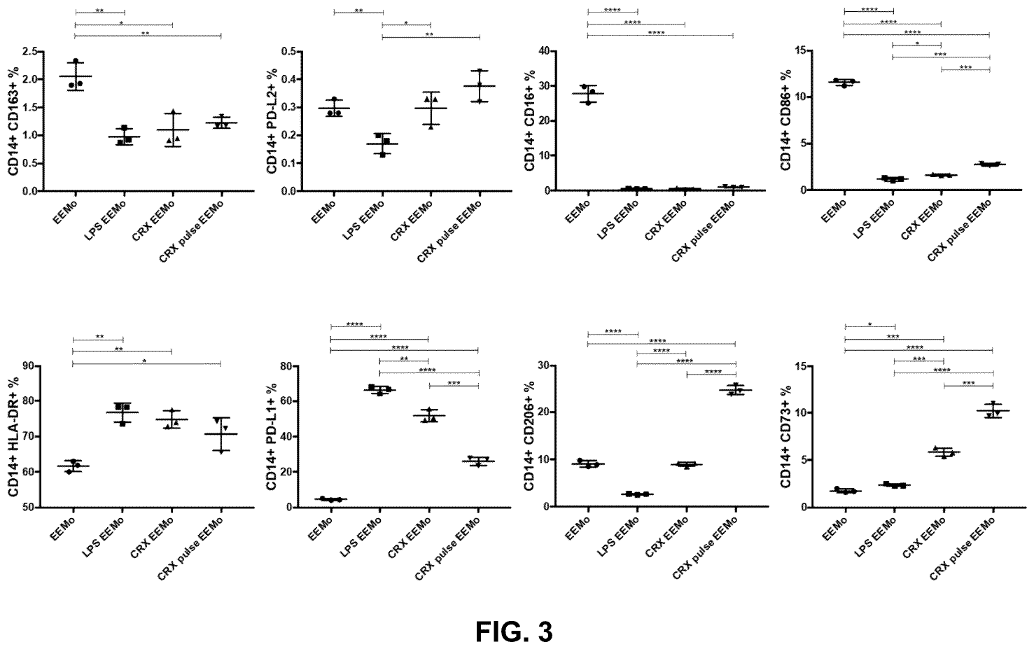

CRX-EVs can be co-cultured with CD14⁺ cells to generate CRX-EEMs, which are immunosuppressive, reparative, anti-inflammatory macrophages. CRX-EVs can be co-cultured with CD14⁺ monocytes to generate CRX-EEMos, which are anti-inflammatory, immunosuppressive, tissue protective, tissue reparative monocytes. When compared by flow cytometry, the external surface markers of CRX-EEMos show a significant increase in the percentage of cells positive for cell surface markers HLA-DR, CD73, and PD-L1 and a significant decrease in expression of cell surface markers CD16, CD163, CD86 compared to control monocytes generated by co-culture with EVs derived from MSCs that have not been primed with a CRX molecule. Additionally, when compared by flow cytometry, external surface markers of CRX-EEMos show a significant increase in the percentage of cells positive for PD-L2, CD86, CD206, and CD73 and a significant decrease in expression of PD-L1 compared to monocytes generated by co-culture with EVs derived from MSCs that have been treated with LPS (LPS-EEMos). See FIG. 3.

Gene expression studies of the CRX-EEMos by qPCR showed statistically significant increases in IL-6, IDO, FGF2, IL-10, IL-12, IL-7, IL-8, IL1B, VEGFA, NFKB, IL-23, IL-13, G-CSF, GM-CSF, CCL-5 MIP-1a and MIP-1b as compared to control monocytes, and a statistical decrease in TGF-ß. See Table 1 and Table 3.

Characteristic surface marker phenotypes and cytokine growth factor profiles of some embodiments of the educated macrophages described herein are outlined in the Examples.

### Treatment

According to the methods of the present disclosure, educated macrophages, educated monocytes, CRX-EVs, CRX, or a combination of any of the foregoing can be administered to a subject in need of thereof. Subjects in need of treatment include those already having or diagnosed with an inflammatory disease or injury as described herein or those who are at risk of developing an inflammatory disease or injury as described herein.

An “inflammatory disease or injury” of the present disclosure can include, but is not limited to, a radiation induced injury or conditions associated with a radiation-induced injury, acute radiation syndrome (ARS), inflammatory diseases (including but not limited to COVID-19), viral or bacterial infections, hyper-immune diseases, autoimmune diseases, trauma associated tissue damage, steroid refractory acute or chronic GVHD, ankylosing spondylitis, atopic dermatitis, bronchopulmonary dysplasia, pulmonary emphysema, myocardial infarction, heart failure, non-ischemic cardiomyopathy, liver allograft rejection, cirrhosis, juvenile idiopathic arthritis, type 1 and type 2 diabetes, acute kidney injury, rheumatoid arthritis, multiple sclerosis, systemic lupus erythematous, osteoarthritis, hemorrhagic stroke, thrombotic stroke, bone fracture, cartilage disorders, tendon, rheumatoid arthritis, Crohn's disease, and ligament disorders, and the like.

Acute radiation syndrome (ARS), also known as radiation toxicity or radiation sickness, can be caused by irradiating the entire body (or most of the body) with a high dose (e.g., greater than about 0.5 gray (Gy) of penetrating radiation over a short period of time (e.g., minutes)). Symptoms of ARS can appear within an hour of exposure and can last for months. The source of radiation exposure can be outside of the subject's body. The penetrating radiation can be high energy x-rays, gamma rays, and neutrons. The types of ARS can include bone marrow syndrome (also referred to as hematopoietic syndrome), gastrointestinal syndrome, and cardiovascular/central nervous system syndrome. The dose of radiation that can cause ARS can be about 0.5 Gy to about 50 Gy, or greater (e.g., 0.1 Gy, 0.2 Gy, 0.3 Gy, 0.4 Gy, 0.5 Gy, 0.7 Gy, 0.8 Gy, 0.9 Gy, 1 Gy, 2 Gy, 3 Gy, 4 Gy, 5 Gy, 6 Gy, 7 Gy, 8 Gy, 9 Gy, 10 Gy, 15 Gy, 20 Gy, 25 Gy, 30 Gy, 35 Gy, 40 Gy, 45 Gy, 50 Gy, or greater). Radiation doses at 3.5-4 Gy can cause death in 50% of people and can be lethal without supportive care.

The severity of ARS can be assessed by repeated complete blood counts (CBCs). CBC tests can determine the levels of red blood cells, white blood cells, neutrophils, lymphocytes, monocytes, and platelets. A decrease in a subject's CBCs indicates that the subject is suffering from ARS. Restoring CBCs to pre-radiation levels is an indication that the subject is recovering from ARS.

With respect to radiation-induced injury, an amount of ionizing radiation exposure resulting in radiation-induced conditions appropriate for treatment or prevention according to a method provided herein is generally between minimal and maximal tolerance doses. The minimal tolerance dose (T/D5/5) is the dose that when administered to a given patient population under a standard set of treatment conditions, results in a rate of severe complications of 5% or less within 5 years of treatment. The maximal tolerance dose (T/D50/5) is the dose that when administered to a given patient population under a standard set of treatment conditions, results in a rate of severe complications of 50% or less within 5 years of treatment. T/D5/5 and T/D50/5 have been established for many conditions and are well-known (see, e.g., Rubin et al. (Eds), 1975, Radiation Biology and Radiation Pathology Syllabus, set RT 1 Radiation Oncology, Chicago, American College of Radiology). The minimal tolerance dose and maximal tolerance dose have been established with respect to therapeutic radiation treatments but are applicable as well for determining the range of radiation exposure suitable for causing the radiation-induced disorders resulting from exposure to radiation from other sources (e.g., occupational or environmental exposures).

Radiation is quantitated on the basis of the amount of radiation absorbed by the body, not based on the amount of radiation produced by the source. A rad (radiation absorbed dose) is 100 ergs of energy per gram of tissue; a gray (Gy) is 100 rad. Radiation dose can be measured by placing detectors on the body surface or by calculating the dose based on radiating phantoms that resemble human form and substance. Radiation dose has three components: total absorbed dose, number of fractions, and time. Most radiation therapy (e.g., external beam radiation therapy, intensity-modulated radiation therapy, TomoTherapy, or proton therapy) programs are fractionated, being delivered in fractions periodically over time, typically once a day, 5 days a week, in 150-200 cGy fractions, generally applied to limited target areas of the body. The total dose delivered in radiation therapy will vary depending on the nature and severity of the condition being treated. For curative cases, the absorbed dose typically will range from 20-80 Gy. For preventative cases, doses are typically around 45-60 Gy and are applied in fractions of about 1.8-2 Gy per day. When used for radiation therapy, ionizing radiation can be provided over a period of time or until a particular amount of radiation exposure has been reached by the target area of the subject. The fractions and dose per fraction can vary for whole body radiation. Sources of ionizing radiation include electrons, X-rays, gamma rays, spaceflight, and atomic ions.

Exposure of a subject to ionizing radiation can be due to a medical procedure including, but not limited to, radiation therapy to treat certain malignant conditions, e.g., lung or breast cancer; medical procedures such as diagnostic X-rays; or procedures involving administering radioactive medicines. Exposure to ionizing radiation also can result from a nuclear accident or from known or suspected occupational or environmental sources, e.g., various consumer products including, but not limited to, tobacco, combustible fuels, smoke detectors, building materials, terrorist attacks, illness due to space travel, and nuclear bombs.

Radiation-induced injuries appropriate for treatment with the methods of the present disclosure can result from exposure to ionizing radiation in the course of radiation therapy. As used herein, the term “radiation therapy” refers to the medical use of high-energy ionizing radiation to shrink tumors, to control malignant cell growth, or, where appropriate, to treat non-malignant conditions such as thyroid eye disease or pigmented villonodular synovitis. X-rays, gamma rays, and charged particles are types of radiation used for radiation therapy. The radiation can be delivered by a machine outside the body (linear accelerators or LINACs), or it can come from encapsulated radioactive material implanted directly into or adjacent to tumor tissues in the body near cancer cells (internal radiation therapy, also called brachytherapy). Systemic radiation therapy uses radioactive substances, such as radioactive iodine, that travel in the blood and are targeted in some fashion to the cancer cells. Teletherapy is the most common form of radiation therapy. About half of all cancer patients receive some type of radiation therapy sometime during the course of their treatment.

Radiation-induced injuries in different tissues and organs generally follow a similar course after exposure to ionizing radiation, particularly as a consequence of radiation therapy. Depending on the dose of ionizing radiation to which the subject is exposed, the subject experiences an acute response phase that generally occurs days to weeks following exposure to ionizing radiation. The acute response phase typically involves inflammatory components, and, if low dose, in some patients, can resolve within a relatively short time or can be fatal. Depending on the dose of ionizing radiation to which the subject is exposed, the acute phase can be followed by a chronic phase, generally beginning one or more months after exposure. The chronic phase is often characterized by extensive tissue remodeling and fibrosis. Results presented herein suggest that effective treatment of the acute response can mitigate or attenuate the chronic phase. Cancers or tumors that occasionally develop, often many years later, at or near the site of radiation exposure are not intended to be included among the disorders suitable for treatment in the method of the present disclosure. Radiation-induced injuries, particularly those resulting from radiation therapy, are well known and have been observed in a variety of tissues and organs. The radiation-induced injury is not the intended result of the radiation therapy but rather is an unintended, and undesirable, side effect of the exposure of various organs, tissues and body parts to the ionizing radiation used in radiation therapy. The radiation-induced injury can be an injury induced by irradiating any, or multiple, body parts, organs or tissues of the subject, including but not limited to bone marrow, lung, heart, bladder, gastrointestinal tract, large intestine, small intestine, stomach, esophagus, skin, ovaries, testes, urogenital system, kidney, head, neck, pancreas, liver, brain, spinal cord, prostate, vasculature, and muscle. In various aspects the radiation-induced injury can be, but is not limited to one or more of bone marrow failure, radiation pneumonitis, radiation enteritis, radiation enteropathy, radiation enterocolitis, radiation dermatitis, radiation-induced erythema, radiation colitis, radiation proctitis, radiation cystitis, radiation nephritis, radiation esophagitis, radiation pericarditis, radiation-induced cardiac effusion, and radiation-induced cardiac fibrosis. All of these disorders are well known and readily identifiable by competent medical practitioners.

Acute kidney injury (AKI) refers to a rapid decline in kidney function over a short period of time (e.g., over the course of one day, two days, or three days). AKI is also referred to as acute kidney failure or acute renal failure. Unlike kidney failure, AKI can be reversible if treated promptly. Supportive treatments for AKI include hydration or renal replacement therapy. AKI occurs in about 10-15% of hospitalized patients and in more than 50% of ICU patients.

AKI can be caused from different origins, such as rhabdomyolysis, chemotherapy (e.g., cisplatin-induced AKI), radiation exposure, low blood pressure, blood or fluid loss, heart attack, organ failure (e.g., heart failure or liver failure), over use of NSAID pain medication (e.g., ibuprofen, ketoprofen, naproxen), severe allergic reactions, burns, trauma, major surgery, sepsis, cancer (e.g., multiple myeloma, vasculitis, interstitial nephritis, scleroderma, tubular necrosis, glomerulonephritis, vasculitis, thrombotic microangiopathy, blockage of the urinary tract (e.g., caused by bladder cancer, prostate cancer, cervical cancer, enlarged prostate, kidney stones, or blood clots), or other processes involving oxidative damage to kidneys.

Graft-versus-host-disease (GVHD) can be characterized by inflammation in different organs. GVHD is a condition that can occurs after an allogeneic hematopoietic stem cell transplantation (also known as a bone marrow transplantation). GVHD can also occur following a blood transfusion if the blood products are not properly treated beforehand. GVHD can also occur after solid organ transplant. In GVHD, the transplanted cells view the transplant recipient cells as foreign and attack them. In other words, with GVHD, donor white blood cells (the graft) can attack the transplant recipient's healthy cells (the host) as if they were a foreign body. GVHD is different than a transplant rejection, which can occur when the immune system of the transplant recipient rejects the transplanted organ, tissue, or cells.

GVHD can be acute or chronic. Acute GVHD can develop in the early weeks and months after a transplant (e.g., within the first 100 days of the transplant). Chronic GVHD can develop about 100 to 600 days post-transplant. A subject can acquire both acute and chronic GVHD at different times or at the same time. Acute or chronic GVHD can also be identified based on clinical presentation of symptoms regardless of the timeframe.

As used herein, the terms “treat” and “treating” refer to therapeutic measures, wherein the object is to slow down or alleviate (lessen) an undesired physiological change or pathological disorder resulting from an inflammatory disease or injury as described herein. For purposes of this disclosure, treating the inflammatory disease or injury includes, without limitation, alleviating one or more clinical indications, decreasing inflammation, reducing the severity of one or more clinical indications of the inflammatory disease or injury, diminishing the extent of the condition, stabilizing the subject's inflammatory disease or injury (i.e., not worsening), delay or slowing, halting, or reversing the disease or injury and bringing about partial or complete remission of the inflammatory disease or injury. Treating the inflammatory disease or injury can also include prolonging survival by days, weeks, months, or years as compared to prognosis if treated according to standard medical practice not incorporating treatment with educated macrophages, educated monocytes, CRX-EVs, or CRX, or combinations thereof, as described herein.

Subjects in need of treatment can include those already having or diagnosed with an inflammatory disease or injury as described herein as well as those prone to, likely to develop, or suspected of having an inflammatory disease or injury as described herein. Pre-treating or preventing a disease or injury according to a method of the present disclosure includes initiating administration of a therapeutic (e.g., human educated macrophages) at a time prior to the appearance or existence of the inflammatory disease or injury, or prior to the exposure of a subject to factors that induce the inflammatory disease or injury. Pre-treating the disorder is particularly applicable to subjects at risk of having or acquiring the disease injury.

Factors that induce inflammatory disease or injury can include any of causes of inflammatory diseases or injuries that are described herein. For example, factors that can induce acute radiation syndrome or radiation injury include, but are not limited to exposure (e.g., full body or most of the body) to a large dose, repeated dose, or prolonged dose of penetrating ionizing radiation. Factors that can induce GVHD include T cells in a donor allogenic transplant that can be further worsened by the conditioning regimen, source of graft tissue, and cell dose. Factors that can induce AKI include, but are not limited to, rhabdomyolysis, chemotherapy (e.g., cisplatin-induced AKI), radiation exposure, low blood pressure, blood or fluid loss, heart attack, organ failure (e.g., heart failure or liver failure), over use of NSAID pain medication (e.g., ibuprofen, ketoprofen, naproxen), severe allergic reactions, burns, injury, major surgery, sepsis, cancer (e.g., multiple myeloma, vasculitis, interstitial nephritis, scleroderma, tubular necrosis, glomerulonephritis, vasculitis, thrombotic microangiopathy, or blockage of the urinary tract (e.g., caused by bladder cancer, prostate cancer, cervical cancer, enlarged prostate, kidney stones, or blood clots).

As used herein, the terms “prevent” and “preventing” refer to prophylactic or preventive measures intended to inhibit undesirable physiological changes or the development of a disorder or condition resulting in the disease or injury. In exemplary embodiments, preventing the disease or injury comprises initiating administration of a therapeutic (e.g., educated macrophages, educated monocytes, CRX-EVs, or a CRX molecule) at a time prior to the appearance or existence of the disease or injury such that the disease or injury, or its symptoms, pathological features, consequences, or adverse effects do not occur. In such cases, a method of the disclosure for preventing the inflammatory disease or injury comprises administering educated macrophages, educated monocytes, CRX-EVs, or a CRX molecule, or combinations thereof to a subject in need thereof prior to exposure of the subject to factors that influence the development of the disease or injury. In some embodiments, preventing the inflammatory disease or injury in the subject comprises administering educated macrophages, educated monocytes, CRX-EVs, or a CRX molecule, or combinations thereof to a subject in need thereof after the subject is exposed to factors that influence the development of the inflammatory disease or injury but before the onset of symptoms associated with the inflammatory disease or injury.

As used herein, the terms “subject” or “patient” are used interchangeably and can encompass any vertebrate including, without limitation, humans, mammals, reptiles, amphibians, and fish. However, advantageously, the subject or patient is a mammal such as a human, or a mammal such as a domesticated mammal, e.g., dog, cat, horse, and the like, or livestock, e.g., cow, sheep, pig, and the like. In exemplary embodiments, the subject is a human. As used herein, the phrase “in need thereof’ indicates the state of the subject, wherein therapeutic or preventative measures are desirable. Such a state can include, but is not limited to, subjects having a disease or injury as described herein or a pathological symptom or feature associated with a disease or injury as described herein.

In some cases, a method of treating or preventing an inflammatory disease or injury as described herein comprises administering a pharmaceutical composition comprising a therapeutically effective amount of educated macrophages, educated monocytes, CRX-EVs, a CRX molecule, or a combination thereof as a therapeutic agent (i.e., for therapeutic applications). As used herein, the term “pharmaceutical composition” refers to a chemical or biological composition suitable for administration to a mammal. Examples of compositions appropriate for such therapeutic applications include preparations for parenteral, subcutaneous, transdermal, intradermal, intramuscular, intracoronarial, intramyocardial, intraperitoneal, intravenous or intraarterial (e.g., injectable), intra-articular, or intratracheal administration, such as sterile suspensions, emulsions, and aerosols. Intratracheal administration can involve contacting or exposing lung tissue, e.g., pulmonary alveoli, to a pharmaceutical composition comprising a therapeutically effective amount of educated macrophages, educated monocytes, CRX-EVs, or a CRX molecule, either alone or in combination. In some cases, pharmaceutical compositions appropriate for therapeutic applications can be in admixture with one or more pharmaceutically acceptable excipients, diluents, or carriers such as sterile water, physiological saline, glucose or the like. For example, educated macrophages, educated monocytes, CRX-EVs, or a CRX molecule described herein can be administered to a subject as a pharmaceutical composition comprising a carrier solution.

Formulations can be designed or intended for oral, rectal, nasal, topical or transmucosal (including buccal, sublingual, ocular, vaginal and rectal) and parenteral (including subcutaneous, intramuscular, intravenous, intraarterial, intradermal, intraperitoneal, intrathecal, intraocular and epidural), inhalational or intratracheal administration. In general, aqueous and non-aqueous liquid or cream formulations are delivered by a parenteral, oral or topical route. In other embodiments, the compositions can be present as an aqueous or a non-aqueous liquid formulation or a solid formulation suitable for administration by any route, e.g., oral, topical, buccal, sublingual, parenteral, aerosol, a depot such as a subcutaneous depot or an intraperitoneal or intramuscular depot. In some cases, pharmaceutical compositions are lyophilized. In other cases, pharmaceutical compositions as provided herein contain auxiliary substances such as wetting or emulsifying agents, pH buffering agents, gelling or viscosity enhancing additives, preservatives, flavoring agents, colors, and the like, depending upon the route of administration and the preparation desired. The pharmaceutical compositions can be formulated according to conventional pharmaceutical practice (see, e.g., Remington: The Science and Practice of Pharmacy, 20th edition, 2000, ed. A. R. Gennaro, Lippincott Williams & Wilkins, Philadelphia, and Encyclopedia of Pharmaceutical Technology, eds. J. Swarbrick and J. C. Boylan, 1988-1999, Marcel Dekker, New York).

The preferred route of administration can vary with, for example, the subject's pathological condition or weight or the subject's response to therapy or that is appropriate to the circumstances. The formulations can also be administered by two or more routes, where the delivery methods are essentially simultaneous or they can be essentially sequential with little or no temporal overlap in the times at which the composition is administered to the subject.

Suitable regimes for initial administration and further doses or for sequential administrations also are variable, can include an initial administration followed by subsequent administrations, but nonetheless, can be ascertained by the skilled artisan from this disclosure, the documents cited herein, and the knowledge in the art.

In some embodiments, a method of treating or preventing an inflammatory disease or injury comprises administering a single dose of a therapeutically effective amount of educated macrophages (CRX-EEMs, CRX-Ms), educated monocytes (CRX-EEMos, CRX-Mos), CRX-EVs, or a CRX molecule, or a combination thereof to a subject in need thereof. For example, educated macrophages, educated monocytes, CRX-EVs, or a CRX molecule, or a combination thereof can be administered in a single dose about 4 hours to about 24 hours (e.g., 4 hours, 5 hours, 6 hours, 7 hours, 8 hours, 9 hours, 10 hours, 12 hours, 14 hours, 16 hours, 18 hours, 20 hours, 22 hours, or 24 hours) after exposure to a high dose of radiation in order to treat and prevent ARS.

In other embodiments, a method of treating or preventing an inflammatory disease or injury comprises administering multiple doses (e.g., 2, 3, 4, 5, 6, 7, 8, 9, or 10 doses, or more) of a therapeutically effective amount of educated macrophages (CRX-EEMs, CRX-Ms), educated monocytes (CRX-EEMos, CRX-Mos), CRX-EVs, or a CRX molecule, or a combination thereof in certain dosage intervals (e.g., days between doses, weeks between doses, months between doses, years between doses).

In some embodiments, a therapeutically effective amount of educated macrophages, educated monocytes, CRX-EVs, or a CRX molecule, or a combination thereof, can be administered to the subject in dosing intervals prior to an anticipated or expected exposure to a factor that induces an inflammatory disease or injury, following exposure to a factor that induces an inflammatory disease or injury, or following the onset of symptoms or diagnosis of an inflammatory disease or injury. The intervals can be early intervals (e.g., on 0, 1, 2, 3, 4, 5, and/or 6 days post-exposure) or late intervals (e.g., on 0, 7, 8, 9, 10, 11, 12, 13, 14, 15, 16, 17, 18, 19, 20, 21, 22, 23, 24, and/or 25 days post-exposure). The dosing intervals can also be continuous and evenly spaced (e.g., every day, every other day, every third day, every week, every other week, every third week, once a month, twice a month, three times a month, every month, every other month, every three months, every four months, or once a year) until the inflammatory disease or injury resolves or enters remission.

For example, educated macrophages, educated monocytes, CRX-EVs, or a CRX molecule, or a combination thereof can be administered to a subject to suppress GVHD either concurrently with a transplant (e.g., separate administrations), combined with the to-be transplanted stem cell product (e.g., in a single administration), or before or after a transplant on the same day as the transplant (day 0), and then again in early intervals on days 1, 3, and 5 post-transplant, on days 2, 4, and 6 post-transplant, or on days 3 and 5 post-transplant. In another embodiment, educated macrophages, educated monocytes, CRX-EVs, or a CRX molecule, or a combination thereof can be administered to a subject to suppress GVHD either concurrently with a transplant, or before or after a transplant on day 0, and then again in late intervals on days 7, 14, and 21 post-transplant, on days 8, 16, and 24 post-transplant, on days 9, 18, and 27 post-transplant, or on days 7, 10, 13, 15, 18, and 21 post-transplant. In other embodiments, educated macrophages, educated monocytes, CRX-EVs, or a CRX molecule, or a combination thereof can be administered to a subject to suppress GVHD in a continuous interval until the symptoms resolve on, for example, either concurrently with the transplant or before or after a transplant on day 0, and then again on days 3, 5, 7, 9, 11, 13, and 15 post-transplant, days 2, 4, 6, 8, 10, 12, 14, 16, 18, 20, 22 post-transplant, or days 1, 2, 3, 4, 5, 6, 7, 8, 9, 10, 11, 12, 13, 14, 15, 16, 17, 18, 19, and 20 post-transplant,

Educated macrophages, educated monocytes, CRX-EVs, or a CRX molecule, or combinations thereof, can be administered to treat AKI in the days, weeks, months, or years following exposure to a factor that can cause AKI or following the onset of symptoms or diagnosis of AKI. In another embodiment, educated macrophages, educated monocytes, CRX-EVs, a CRX molecule or combinations thereof can be administered on the same day that the subject is exposed to a factor that can cause AKI, such as chemotherapy or a heart attack in the subject, either concurrently with or prior to or after exposure to the factor.

In some embodiments, educated macrophages, educated monocytes, CRX-EVs, a CRX molecule or combinations thereof can be administered as a prophylactic in the days, weeks, months, or years prior to exposure to a factor that causes an inflammatory disease or injury. For example, a subject can be administered educated macrophages, educated monocytes, CRX-EVs, CRX or combinations thereof in the days (e.g., 1 day, 2 days, 3 days, 4 days, 5 days, 6 days, or 7 days) prior to an allogeneic transplant (e.g., bone marrow transplant or peripheral blood stem cell transplant) to prevent or suppress the onset of GVHD.

In some embodiments, educated macrophages, educated monocytes, CRX-EVs, a CRX molecule or combinations thereof can be administered to treat one or more different inflammatory disease at the same time. For example, educated macrophages, educated monocytes, CRX-EVs, a CRX molecule or combinations thereof can be administered to a subject that has undergone full body radiation in preparation for a bone marrow transplant in order to treat or prevent (1) a radiation injury and (2) GVHD concurrently. As another example, educated macrophages, educated monocytes, CRX-EVs, a CRX molecule or combinations thereof can be administered to a subject that has undergone full body radiation in preparation for a bone marrow transplant in order to treat or prevent (1) COVID-19 and (2) AKI concurrently.

In some embodiments, the methods of treating or preventing an inflammatory disease or injury can comprise administering a combination of two or more of CRX-EEMos, CRX-EEMs, CRX-Mos, CRX-Ms, CRX-EVs, and a CRX molecule, including but not limited to administering: (1) CRX-EEMos and CRX-EVs; (2) CRX-EEMs and CRX-EVs; (3) CRX-EMos and CRX-EV; (4) CRX-EMs and CRX-EV; (5) CRX-EEMos, CRX-EVs, a CRX molecule; (6) CRX-EMos, CRX-EV, and a CRX molecule; (7) CRX-Mos and CRX-EV; or (8) one or more CRX molecules (e.g., CRX-527 and CRX-555). Any combination of these elements is contemplated by the disclosure.

In some cases, educated macrophages, educated monocytes, CRX-EVs, a CRX molecule or combinations thereof can optionally be administered in combination with one or more additional active agents, including exosomes or microvesicles. Such active agents include anti-inflammatory, anti-cytokine, analgesic, antipyretic, antibiotic, immunosuppressive agents and antiviral agents, as well as growth factors and agonists, antagonists, and modulators of immunoregulatory agents (e.g., TNF-α, IL-2, IL-4, IL-6, IL-10, IL-12, IL-13, IL-18, IFN-α, IFN-γ, BAFF, CXCL13, IP-10, VEGF, EPO, EGF, HRG, Hepatocyte Growth Factor (HGF), Hepcidin, including antibodies reactive against any of the foregoing, and antibodies reactive against any of their receptors). Any suitable combination of such active agents is also contemplated. When administered in combination with one or more active agents, educated macrophages can be administered either simultaneously or sequentially with other active agents. For example, victims of ARS can simultaneously receive educated macrophages, educated monocytes, CRX-EVs, or a CRX molecule and a growth factor (such as G-CSF or PEG-G-CSF), a cytokine (such as IL-3, IL-11, IL-12), a population of cells (such as lymphoid or myeloid progenitors), or a small molecule radio-protective agent (such as amifostine or genistein) for a length of time or according to a dosage regimen sufficient to support recovery and to treat, alleviate, or lessen the severity of the radiation injury. In some embodiments, educated macrophages, educated monocytes, CRX-EVs, or a CRX molecule of the present disclosure can also be administered to a patient simultaneously with or prior to receiving a radiation treatment, such as a treatment for cancer. In some embodiments, CD14⁺ monocytes or macrophages are administered simultaneously with CRX-EVs and/or a CRX molecule to a patient.

In some embodiments, educated macrophages, educated monocytes, CRX-EVs, CRX, or a combination thereof are administered to a human or non-human subject in need thereof using an infusion, topical application, surgical transplantation, co-infusion with an organ, tissue or stem cell product, or implantation and direct injection into an organ or tissue. In exemplary embodiments, administration is systemic. In such cases, educated macrophages, educated monocytes, CRX-EVs, a CRX molecule, or a combination thereof can be provided to a subject in need thereof in a pharmaceutical composition adapted for intravenous administration to subjects. Compositions for intravenous administration can be solutions in sterile isotonic aqueous buffer. The use of such buffers and diluents is well known in the art. Where necessary, the composition can also include a local anesthetic to ameliorate any pain at the site of the injection. Generally, the ingredients are supplied either separately or mixed together in unit dosage form, for example, as a cryopreserved concentrate in a hermetically sealed container such as an ampule indicating the quantity of active agent. Where the composition is to be administered by infusion, it can be dispensed with an infusion bottle containing sterile pharmaceutical grade water or saline. Where the composition is administered by injection, an ampule of sterile water for injection or saline can be provided so that the ingredients can be mixed prior to administration. In some cases, compositions comprising human educated macrophages, educated monocytes, CRX-EVs, a CRX molecule or combinations thereof are cryopreserved prior to administration.

Therapeutically effective amounts of educated macrophages, educated monocytes, CRX-EVs, a CRX molecule, or combinations thereof are administered to a subject in need thereof. An effective dose or amount is an amount sufficient to effect a beneficial or desired clinical result. With regard to methods of the present disclosure, the effective dose or amount, which can be administered in one or more administrations, is the amount of human educated macrophages, educated monocytes, CRX-EVs, or a CRX molecule sufficient to elicit a therapeutic effect in a subject to whom the cells are administered. In some cases, an effective dose of educated macrophages or educated monocytes is about 1×10⁵ cells/kilogram to about 10×10⁹ cells/kilogram (e.g., 1×10⁵ cells/kilogram, 1×10⁶ cells/kilogram, 1×10⁷ cells/kilogram, 1×10⁸ cells/kilogram, or 1×10⁹ cells/kilogram) of body weight of the recipient. In some cases, an effective dose of CRX-EVs is about 1×10⁵ EV/kilogram to about 10×10¹⁰ EV/kilogram (e.g., 1×10⁵ EV/kilogram, 1×10⁶ EV/kilogram, 1×10⁷ EV/kilogram, 1×10⁸ EV/kilogram, 1×10⁹ EV/kilogram, or 1×10¹⁰ EV/kilogram) body weight of the recipient. Effective amounts will be affected by various factors that modify the action of the cells upon administration and the subject's biological response to the cells, e.g., severity of radiation injury, type of damaged tissue, the patient's age, sex, and diet, time of administration, and other clinical factors.

Therapeutically effective amounts for administration to a human subject can be determined in animal tests and any art-accepted methods for scaling an amount determined to be effective for an animal for human administration. For example, an amount can be initially measured to be effective in an animal model (e.g., to achieve a beneficial or desired clinical result). The amount obtained from the animal model can be used in formulating an effective amount for humans by using conversion factors known in the art. The effective amount obtained in one animal model can also be converted for another animal by using suitable conversion factors such as, for example, body surface area factors. If CRX were to be administered directly to a subject as a therapeutic agent to treat an inflammatory or tissue disease, a suitable dose is expected to be in the range of 1-100 ng/kg, or more preferably 10-100 ng/kg. In some embodiments, the dose of a CRX molecule administered directly to a subject can be 1 ng/kg, 5 ng/kg, 10 ng/kg, 15 ng/kg, 20 ng/kg, 25 ng/kg, 30 ng/kg, 35 ng/kg, 40 ng/kg, 45 ng/kg, 50 ng/kg, 55 ng/kg, 60 ng/kg, 65 ng/kg, 70 ng/kg, 75 ng/kg, 80 ng/kg, 85 ng/kg, 90 ng/kg, 95 ng/kg, or 100 ng/kg.

It is to be understood that, for any particular subject, specific dosage regimes can be adjusted over time according to the individual need and the professional judgment of the person administering or supervising administration of the educated macrophages, educated monocytes, CRX-EVs, or a CRX molecule. For example, an educated macrophage or CRX-EV dosage for a particular subject with a radiation injury can be increased if the lower dose does not elicit a detectable or sufficient improvement in one or more symptoms of radiation injury. Conversely, the dosage can be decreased if the radiation injury is treated or eliminated.

In some cases, therapeutically effective amounts of educated macrophages, educated monocytes, CRX-EVs, a CRX molecule, or combinations thereof can be determined by, for example, measuring the effects of a therapeutic in a subject by incrementally increasing the dosage until the desired symptomatic relief level is achieved. A continuing or repeated dose regimen can also be used to achieve or maintain the desired result. Any other techniques known in the art can be used as well in determining the effective amount range. The specific effective amount will vary with such factors as the particular disease state being treated, the physical condition of the subject, the type of animal being treated, the duration of the treatment, and the nature of any concurrent therapy.

Following administration of educated macrophages, educated monocytes, CRX-EVs, a CRX molecule, or combinations thereof to a subject afflicted by, prone to, or likely to develop an inflammatory disease or injury described herein, a clinical symptom or feature associated with the inflammatory disease or injury can be observed and assessed for a positive or negative change. For example, for methods of radiation injury in a subject, positive or negative changes in the subject's inflammation, infection, bleeding or anemia during or following treatment can be determined by any measure known to those of skill in the art including, without limitation, blood counts.

In any of the methods of the present disclosure, the donor and the recipient of the educated macrophages, educated monocytes, or CRX-EVs can be a single individual, autologous, or different individuals, for example, allogeneic or xenogeneic individuals. Stromal cells and CD14⁺ cells for use in the present disclosure do not need to be from the same donor, patient or source. As used herein, the term “allogeneic” refers to something that is genetically different although belonging to or obtained from the same species (e.g., allogeneic tissue grafts or organ transplants). “Xenogeneic” means the cells are derived from a different species. In one embodiment, CD14⁺ cells can be collected from patients, educated, and then given afresh to a person following or concurrently with radiation treatment such as a cancer treatment. In some embodiments, any allogeneic donor can act as a universal third party donor of CD14⁺ cells.

The present disclosure has been described in terms of one or more preferred embodiments, and it should be appreciated that many equivalents, alternatives, variations, and modifications, aside from those expressly stated, are possible and within the scope of the disclosure.

### EXAMPLES

The embodiment described herein demonstrates using CRX-EEMos or direct co-culture with CRX to treat radiation-induced injury. The embodiment described here also demonstrates the use of EVs from MSCs after CRX priming for treating radiation-induced injury.

### Materials and Methods for Examples 1-6

Cell culture—Monocytes were isolated from human peripheral blood using magnetic bead separation methods according to manufacturers' protocols. Briefly, peripheral blood mononuclear cells were collected from blood from healthy donors after mobilization by density gradient separation using Ficoll-Paque Plus (endotoxin tested) (GE Healthcare Bio-Sciences, Piscataway, NJ, USA) using an IRB-approved protocol. If peripheral blood has undergone apheresis designed to concentrate white cells and exclude red blood cells the density gradient separation step can be skipped, in which case the cells are diluted in buffer such as phosphate-buffered saline (PBS), centrifuged at 300-1000×g and the pellet resuspended in ACK lysis buffer. Red blood cells were lysed by incubating cells in ACK lysis buffer for 3-5 minutes and mononuclear cells were washed with PBS (Hyclone, Logan, UT, USA). To reduce platelet contamination, cell suspensions were centrifuged at 300-700 rpm for 10 minutes and cell pellets were re-suspended in Miltenyi separation buffer with anti-human CD14 microbeads as directed by the manufacturer (Miltenyi Biotec, Auburn, CA, USA) and incubated for 15 minutes at 4° C. After washing to remove unbound antibody, cell separation was achieved using an autoMACS Pro Separator (Miltenyi Biotec). Purity of isolated CD14⁺ cells was >95% when checked by flow cytometry. Purified CD14⁺ monocytes were either plated into six-well cell culture plates at about 0.5-1×10⁶ cells per well for characterization studies or 10⁷ per T75 cm² filter cap cell culture flask for animal studies (Greiner Bio-One, Monroe, NC, USA) in Iscove's modified Dulbecco's media (Gibco, Life Technologies, Grand Island, NY) supplemented with 10% human serum blood type AB (Mediatech, Herndon, VA, USA or Valley Biomedical Inc, Winchester, VA, USA), 1× nonessential amino acids (Lonza, Walkersville, MD, USA), 4 mM L-glutamine (Invitrogen, Carlsbad, CA, USA), 1 mM sodium pyruvate (Mediatech), and 4 μg/mL recombinant human insulin (Invitrogen). Cells were cultured for 7 days at 37° C. with 5% CO₂, without cytokines, to allow differentiation to macrophages. Attached cells were harvested using Accumax dissociation media (Innovative Cell Technologies, Inc, San Diego, CA).

MSCs were isolated from filters left over after bone marrow (BM) harvest from normal healthy donors using an IRB-approved protocol. Briefly, BM cells trapped in the filter were recovered by rinsing the filter with PBS and mononuclear cells were separated using Ficoll-Hypaque 1.073 (GE Healthcare Bio-Sciences). Red blood cells were lysed with 3-minute incubation in ACK lysis buffer (Lonza, Walkersville, MD, USA) and mononuclear cells were suspended in α-minimum essential medium supplemented with 10% fetal bovine serum (US origin, uncharacterized; Hyclone, Logan, UT, USA), 1×nonessential amino acids, and 4 mM L-glutamine. Cells were cultivated in 75-cm² filter cap cell culture flasks. Attached cells (passage 0) were harvested using TrypLE™ cell dissociation enzyme (Invitrogen) and then re-plated into new flasks. Passage 4-6 cells were used for characterization studies and used to isolated EV. MSC identity was confirmed by morphology, adherence to plastic, and flow cytometry. Their immunomodulatory properties on T-cell proliferation were confirmed by an immunopotency assay (Bloom et al., 2015, Cytotherapy, 17:2 140-151).

Isolating and characterizing EVs from cells—Cells (MSCs or macrophages) in 75-cm² filter cap cell culture flasks were washed once with PBS, and the medium was replaced with StemPro® MSC serum-free media (SFM) CTS (A103332-01, Gibco Life Technologies). Cells were incubated for 18-24 hours and the conditioned culture media (CM) was collected. Control exosomes from unprimed MSCs served as exosome controls (MSC-exosomes). To prime MSCs, the MSCs were cultured in SFM supplemented either with 1.0 μg/ml E. coli lipopolysaccharide (LPS) 0111:B4 (L4391 Sigma, St Louis, MO, USA) or with 100 ng/ml synthetic lipid A CRX-527 (“CRX,” InvivoGen, San Diego, CA). All EVs secreted into the SFM from MSCs during the incubation were isolated by a 2-step centrifugation process as described (Thery, C. et al., 2006, Current Protocols in Cell Biology 3.22.1-3.22.29). Briefly, CM from each culture was centrifuged at 2000×g at 4° C. for 20 minutes using an Allegra© X-15R centrifuge (Beckman Coulter, Indianapolis IN, USA) to remove detached cells, apoptotic bodies and cell debris. Clarified supernatant CM was centrifuged in an Optima™ L-80XP Ultracentrifuge (Beckman Coulter) at 100,000×g avg at 4° C. for 2 hours with a SW 28 rotor to pellet exosomes. The supernatant was carefully removed, and EV-containing pellets were re-suspended in PBS and pooled. EV pellets were typically resuspended in 30 to 100 μl PBS/10 ml of CM to yield about 10¹⁰-1¹ EV particles/ml. After re-suspension, the EVs were aliquoted and stored frozen at −80° C. before use. To visualize the EVs by transmission electron microscopy (TEM), the re-suspended EVs were layered on a 30% sucrose cushion and re-centrifuged for 2 hours at 100,000 gavg at 4° C. The upper portion of the cushion was collected and re-centrifuged. The pellet was resuspended in a small volume of PBS, whole-mounted on Formvar EM grids, and stained with uranyl acetate.

The collected EV were designated as follows: EV from MSCs not cultured with LPS or CRX (MSC-EV), MSCs cultured for 24 hours with CRX (CRX-EV) or with LPS (LPS-EV), and MSCs cultured for 2 hours with CRX, washed with PBS wash to essentially remove any free CRX, followed by continued incubation in SFM for 22 hours (CRX-pulse-exosomes).

Characterizing EV (exosome) surface marker profile by MACSPlex—The surface marker profile of EVs from two MSC isolates of both unstimulated MSC-EVs and CRX-EVs were determined by flow cytometry using the MACSPlex Exosome Kit (Miltenyi Biotec). This kit permits detection of 37 exosomal surface markers (DIc, CD2, CD3, CD4, CD8, CD9, CD11c, CD14, CD19, CD20, CD24, CD25, CD29, CD31, CD40, CD41b, CD42a, CD44, CD45, CD49e, CD56, CD62P, CD63, CD69, CD81, CD86, CD105, CD133/1, CD142, CD146, CD209, CD326, HLA-ABC, HLA-DRDPDQ, MCSP, RORI, SSEA-4), and two isotype markers that served as isotype controls (REA isotype control, and mlgG1 isotype control). This assay was performed according to manufacturer's protocol. In brief, capture beads coupled with antibodies to the exosome surface markers were mixed with equal volumes of purified MSC-exosomes and gently rotated in the dark at 4° C. overnight. The bead-exosome complexes were washed and then incubated for 1 hour with detection bead mixture consisting of pan-exosome markers CD9, CD63, and CD81 labeled with FITC, PE or APC. The beads were then washed and resuspended in 150 μl of MACSPlex buffer for analysis. Prior to experimentation, the system was calibrated and background settings were adjusted to unlabeled beads. The auto-sampler used 100 μl from each sample to collect beads and automated gating strategies were used to identify bead populations for each analyte. Batch analysis quantified median intensities for each bead population and analyte surface expression was calculated for each sample. Miltenyi MACSQuant Analyzer 10 for sample acquisition and MACSQuantify Software was used for data analysis. MFI for each surface marker was determined from exosomes isolated from different MSC isolates and then averaged. MFI values were compared to background from isotype controls and values of 1 or more were considered significant and positive for the presence of the surface marker.

Educating CD14⁺ cells using EVs—To educate CD14⁺ monocytes, frozen stocks of CD14⁺ monocytes were thawed then placed in the supplemented Iscove's medium described, supra. Within 1 hour, the thawed monocytes were educated with EVs for 18-24 hours. To educate CD14⁺ macrophages, frozen stocks of CD14⁺ monocytes were thawed then placed in complete macrophage/monocyte medium and allowed to differentiate to adherent macrophages for 5-7 days. These macrophages were placed into fresh medium and educated with EVs for 3 additional days. EV particle concentration, typically 10¹⁰ to 10¹¹ particles/ml in PBS, was determined using the IZON qNano Nanoparticle Characterization instrument (Cambridge, MA, USA). The amount of EV preparation used for education was based on dose-response studies using EVs coupled with flow-cytometry to determine changes in surface marker expression. Monocytes were seeded into 6 well plates at 1×10⁷ cells/well (2 mls media) or in 75-cm² filter cap cell culture flasks (10 mls of media). The seeded monocytes were educated with 1-5×10⁹ EVs (MSC-EVs, LPS-EVs or non-MSC control EVs obtained from Day 10 macrophages.

Educated macrophages were generated by treating macrophages with MSC-EV to produce EEMs, LPS-EEMs. Direct co-culture of CD14⁺ macrophages with MSCs generated MSC-educated macrophages (MEMs). Day 7 macrophages were supplemented with fresh media and incubated with human BM-derived MSCs at an approximate macrophage:MSC ratio of 10:1, and incubated for 3 days to generate MEMs.

Educating CD14⁺ cells ex vivo with CRX. For CRX treatment of monocytes, approximately 1×10⁷ cells in 10 mls of media were treated with 1 pg of CRX-527 (100 ng CRX/ml) in macrophage/monocyte culture media, incubated for 24 hours, then washed with PBS by centrifugation. Monocytes directly treated with CRX were designated as CRX-educated monocytes (CRX-EMos)

Educating cells in vivo by direct treatment with CRX-EV or LPS-EV. Approximately 5×10⁹ MSC-exosomes, CRX-EV, CRX-pulse-exosomes, or LPS-exosomes were administered to each mouse. Exosome stocks were thawed from −80° C. before use and were suspended and diluted to a volume of 100-200 μl PBS followed by i.v. (intravenous, parenterally) treatment in the mice.

Flow Cytometry—EEMos, LPS-EEMos, CRX-EEMos, and CRX-pulse-EEMos were collected at day 2 of culture, counted, and incubated with Fc block (BD Pharmingen, San Jose, CA, USA, cat #: 564220) for 10 minutes at room temperature and then stained at 4° C. for 20-30 minutes with anti-human antibodies in staining buffer (PBS with 2% FBS). The cells were then washed with PBS, centrifuged at 300 g for 10 minutes, and Ghost Dye™ Red 780 viability dye (Tonbo Biosciences, cat #13-0865) was added for 20 minutes at room temperature. Cells were then washed with staining buffer, spun down, and resuspended in 400 μl of staining buffer. Cells were then run on an Attune™ NxT flow cytometer (Thermo Fisher Scientific) and analyzed using Flowjo™ software (BD). All antibodies were purchased from BioLegend (San Diego, CA). Antibodies included: CD206-PE: (15-2, cat #321105), CD163-FITC: (GHI/61, cat #333617), PD-L1-BV711: (29E.2A3, cat #329721), PD-L2-APC: (24F.10C12, cat #329608), CD14-BV421: (HCD14, cat #325627), CD16-AF700: (3G8, cat #302025), HLA-DR-BV605: (L243, cat #307639), CD73-PE/Cy7: (TY/11.8, cat #127223), and CD86-BV510: (IT2.2, cat #305431).

Secretome analysis—Monocytes from three different human isolates were placed 48 well plates (approximately 2×10⁵ cells/well) in 250 ul of media and educated with MSC exosomes or CRX-exosomes to produce EEMos and CRX-EEMos, respectively or uneducated control monocytes with PBS (Hyclone) for 24 hours. Each isolate and condition was performed in triplicate. After incubation, the culture media was collected, centrifuged at 300×g for 10 minutes to remove any floating cell debris and supernatant assayed for secreted factors. Additional assay controls included culture media spiked with control MSC or CRX exosomes. Thirty secreted factors were analyzed using the Cytokine Human Magnetic 30-Plex Panel for the Luminex™ platform (LHC6003M, ThermoFisher Scientific) quantitating human proteins for interleukins: IL-IRA, IL-1 beta, IL-2, IL-2R, IL-4, IL-5, IL-6, IL-7, IL-8, IL-10, IL-12 (p40/p70), IL-13, IL-15, IL-17, interferon alpha (IFNα), interferon-gamma (IFNγ), interferon gamma-induced protein 10 (IP-10), monokine induced by gamma interferon or CXCL9 (MIG), epidermal growth factor (EGF); hepatocyte growth factor (HGF), vascular endothelial growth factor A (VEGF), chemokine ligand 5 (CCL-5), also called regulated upon activation normal T cell expressed and secreted (RANTES), eotaxin, tumor necrosis factor alpha (TNF alpha), fibroblast growth factor 2 (FGF-basic), monocyte chemoattractant protein 1 (MCP-1), macrophage inflammatory protein 1a (MIP-1 alpha), macrophage inflammatory protein 1b (MIP-1 beta), granulocyte-macrophage colony-stimulating factor (GM-CSF) and granulocyte-colony stimulating factor (G-CSF). Analytes in the supernatants were detected at the pg/ml range on a Luminex xMAP platform.

Gene expression analysis—RNA was isolated from cells using an RNeasy™ micro kit (Qiagen, Valencia, CA, USA), and the quality of isolated RNA was determined using Nanodrop 1000 (Fisher Scientific, Pittsburgh, PA, USA). RNA was converted to cDNA using a Quantitect reverse transcription kit (Qiagen). Quantitative polymerase chain reaction (qPCR) was performed using Power SYBR green master mix (Applied Biosystems, Foster City, CA, USA) on StepOne Plus instrument (Applied Biosystems) using standard protocols. Verified primers for IL-10, indoleamine 2,3-dioxygenase (IDO), IL-1B, IL-6, IL-8, IL-12, IL-23, TGF-ß, VEGF-A, FGF2, IL-7, NF-KB, TGF-α, IL-13, macrophage inflammatory protein 1a (MIP-1 alpha), macrophage inflammatory protein 1b (MIP-1 beta), granulocyte-macrophage colony-stimulating factor (GM-CSF) and granulocyte-colony stimulating factor (G-CSF) and chemokine ligand 5 (CCL-5) were purchased from Qiagen. The threshold cycle (Ct) value for each gene was normalized by the average Ct using the GAPDH housekeeping gene and using this normalization the expression values of the control macrophages or control monocytes were set at 1.

Mice—Male and Female immunocompromised NOD.Cg-Prkdc^{scid} Il2rg^{tm1Wj1}/SzJ (NSG) and immunocompetent ICR (ICR/HaJ) mice were purchased from The Jackson Laboratory (Bar Harbor, Maine) and used at 8-16 weeks of age. All animals were bred and housed in a pathogen-free facility throughout the study. The Animal Care and Use Committee at the University of Wisconsin approved all experimental protocols.

In vivo lethal radiation injury model in immunocompromised mice—On day 0, approximately equal numbers of male and female NSG mice received 4 Gy lethal total body irradiation using an X-RAD 320 X-ray irradiator (Precision X-Ray, North Branford, CT, USA) to induce consistent lethality within a 2-week time frame. For all animal studies, mice were treated four hours after radiation injury intravenously (i.v.) in the tail vein with 100 μl of test material re-suspended in PBS. Treatment with 100 μl PBS served as a control in all animal studies. For studies on treating mice with exosome-educated monocytes, mice were treated with approximately 1×10⁷ EEMos, CRX-EEMos, or CRX-pulse-EEMos. For studies on treating mice with exosomes, mice were treated with approximately 5×10⁹ exosomes with either control MSC-exosomes, LPS-exosomes, CRX-EV, or CRX-pulse-exosomes. For studies on treating mice with monocytes directly stimulated with CRX, mice were treated with approximately 1×10⁷ cells with CRX-educated monocytes (CRX-EMos). The mice were monitored at least 3 times a week for survival and weight change. Clinical scores were also determined based on a modified clinical scoring system for GVHD. Cumulative scores of percent weight loss, posture, activity, and fur texture (scored from 0-2 for each criteria), were recorded as previously described. Animals were considered “protected” when statistical improvement of P≤0.05 or at least a statistical trend (0.06-0.1) in the cumulative clinical score was observed within a period of time and/or overall survival. On days 5 and 30 post-challenge, blood from surviving mice was collected in a microtainer tube K2 EDTA (Becton Dickinson, Franklin Lakes, NJ) or equivalent from a tail vein nick and the hematology of the whole blood was assayed on a Hemavet™ 950FS analyzer (Drew Scientific Inc., Miami Lakes, FL, USA).

In vivo lethal radiation injury model in immunocompetent mice—On day 0, ICR mice received 9 Gy lethal total body irradiation using an X-RAD 320 X-ray irradiator (Precision X-Ray, North Branford, CT, USA). Twenty-four hours after radiation challenge, mice were treated by intravenous administration in the tail vein with 100 ul of exosomes. Specifically for direct exosome treatment, either PBS (Hyclone) (controls), 5×10⁹ MSC exosomes or CRX exosomes was administered. Mice were monitored for survival and clinical scores associated with ARS 3-5 times a week. Clinical scores were determined using a modified clinical scoring system as previously described (Cooke et al., 1996, Blood, 88:3230-323) based on cumulative score of percent weight loss, posture, activity, and fur texture (scored from 0-2 for each criteria). The mice were euthanized if the cumulative clinical score was a six or greater. Complete blood counts (CBC) were performed to determine the pre-radiation (pre-rad controls) status and at strategic intervals post-radiation challenge on surviving mice to determine the effects of radiation and treatments on the CBC. The mice were bled by nicking the tail vein and blood was collected in a microtainer K2 EDTA tube (cat #365974 Becton Dickenson, Franklin Lakes, NJ, USA). Whole blood hematology was assayed on a Hemavet 950FS analyzer (Drew Scientific Inc., Miami Lakes, FL,) at strategic time points early post-challenge on (days 6), the early recovery period, (day 20) and during the late recovery period, (day 41). CBC's were reported as the mean cell number/volume for red blood cells (RBCs), white blood cells (WBC), neutrophils lymphocytes, monocytes, platelets and platelet volume.

Statistical analysis—Statistics were performed using GraphPad Prism version 7.0 (GraphPad Software, San Diego, CA). Data were reported as mean±SEM. For analysis of three or more groups, the analysis of variance (ANOVA) test was performed with the Kruskal-Wallis and Dunn's multiple comparisons post-test. Principal component analysis and t-tests (unequal variance) comparing gene expression between groups were performed on Microsoft Excel. Multiple hypotheses testing correction was done using the Benjamini Hochberg false discovery rate (FDR) procedure. A p-value less than 0.05 was considered statistically significant. Statistical analyses were compared to the normal CBC levels in mice before radiation.

### Example 1: Stimulating MSCs with TLR4 Agonists to Produce Therapeutic EVs

Anti-inflammatory, regenerative cells were produced by stimulating secretion from MSCs of EVs using a synthetic TLR4 agonist. EVs were heterogeneous in size and included small exosomes (50-200 nm) and large microvesicles (400-1000 nm). Described herein is an ex vivo process, where the type of EVs produced by MSCs was directly controlled by stimulating them with a TLR4 agonist. TLR4 agonists such as bacterial lipopolysaccharides (LPS) are typically considered pro-inflammatory, and direct stimulation with LPS usually signals cells to produce cytokines that potentiate more inflammation. Stimulating MSCs with TLR4 agonists promoted an opposite effect, due to the immunomodulatory nature of MSCs. The synthetic TLR4 agonist CRX-527 was used, instead of natural sources of LPS derived from gram negative bacteria because CRX-527 is much less toxic than LPS to humans and possesses superior purity without contaminants.

MSCs were stimulated with CRX, either as a continuous 24-hour treatment or as a 2-hour pulse, and CRX-EV and CRX-pulse-EVs secreted from the MSC were harvested by ultra-centrifugation and characterized for particle size distribution and concentration. The mean- and mode diameters were similar for the CRX-EVs (159+/−22 nm and 116+/−17 nm) and for the CRX-pulse-EVs (162 nm and 117 nm). Mean particle concentrations were also similar for the CRX-EVs (1.9×10¹¹+/−1.7×10¹¹ particles/ml) and the CRX-pulse-EVs (9.2×10¹⁰ particles/ml). The CRX-EV expressed certain markers at levels quite different from EV obtained from MSC not exposed to CRX (MSC-EV), by MACSPlex flow cytometry (FIG. 1). In the CRX-EV, surface markers CD44, CD29 and CD146 were statistically significantly higher than in MSC-EV (FIG. 2). These markers bind with extracellular matrix (ECM) produced during healing of injury. These EVs can be targeted to areas undergoing tissue remodeling. RNA sequencing analysis of CRX-EV and MSC-EV can reveal differences in RNA content between the two and can identify key signal molecules or transcription factors important for protecting cells during radiation injury.

### Example 2: Educating Cells Ex Vivo with CRX-EV

Monocytes were exposed ex vivo to CRX-EV to produce CRX-EEMos. Monocytes were separately exposed to MSC-exosomes to produce control EEMos. CRX-EV polarized monocytes into a unique M2-like phenotype, compared to the EEMos. Gene expression for IL-6, IDO, FGF2, IL-10, IL-12, IL-7, IL-8, IL1B, VEGFA, NF-KB, and IL-23 was significantly higher (P<0.05) in the CRX-EEMos compared to the control monocytes (Table 1). A subset of these genes—IL-6, IL-10, IDO, IL-8, IL-1B, VEGFA and IL-23—was higher in the CRX-EEMos than in the EEMos. (P<0.05). Overall, gene expression was mostly higher in the CRX-EEMos than in the CRX-pulse-EEMos, but statistically so only for IL-1B. Of note, gene expression of pro-inflammatory cytokine IL-12 was two-fold lower for CRX-EEMos than for the CRX-pulse-EEMos.

Statistical significance of mean levels of gene expression by qPCR of monocytes educated with MSC-EVs (EEMos), monocytes educated with 2 hour pulse CRX-EVs (CRX-pulse EEMos) or monocytes educated with CRX-EVs for 18-24 hours (CRX-EEMos) were compared to control monocytes. *P≤0.05, **P≤0.005, ***P≤0.0005, ****P≤0.0001.

TABLE 1
Gene expression of CRX-EEMos by qPCR

| | Control | | CRX-pulse- | |
|---|---|---|---|---|
| Gene | monocytes | EEMos | EEMos | CRX-EEMos |
| IL-6 | 1.0 | 3.9*** | 3126** | 21117* |
| IDO | 1.0 | 30.6**** | 614** | 1011 ** |
| FGF2 | 1.0 | 2.2**** | 277*** | 422** |
| IL-10 | 1.0 | 0.9 | 5.6** | 5.9**** |
| IL-12 | 1.0 | 1.1 | 3.1* | 1.5* |
| IL-7 | 1.0 | 1.6 | 13.5** | 27.1* |
| IL-8 | 1.0 | 5.2 | 97**** | 211.8** |
| IL-1B | 1.0 | 2.8**** | 1553**** | 5586** |
| VEGF-A | 1.0 | 1.0 | 1.0 | 1.7** |
| NFKB | 1.0 | 1.6*** | 1.6 | 1.3** |
| TGF-β | 1.0 | 1.0 | 0.5* | 0.35 **** |
| TNF-α | 1.0 | 1.5 | 1.7* | 1.4 |
| IL-23 | 1.0 | 0.4** | 3.6** | 2.3** |

*P ≤ 0.05,
**P ≤ 0.005,
***P ≤ 0.0005,
****P ≤ .0001

Secretome analysis: Interleukins, chemokines and growth factors that were detected (pg/ml) by multiplex analysis in the culture supernatants from the CRX-EEMos are shown (Table 2). All of the factors shown in Table 2 were secreted by CRX-EEMos. Of these released factors, nine were strongly induced after education and significantly higher in the CRX-EEMos compared to either the untreated control monocytes or the EEMos. There were very significant increases in released levels of interleukins IL-6, IL-10, IL-13 and TNF-α in the CRX-EEMos. (P≤0.0005) In addition, chemotactic chemokines, CCL-5, MIP-1a and MIP-1a were also significantly higher in the CRX-EEMos compared to the controls. Growth factors G-CSF and GM-CSF were also secreted at significantly higher levels by the CRX-EEMos compared to the controls. Some of the elevated factors (IL-6, G-CSF, and GM-CSF) are known to be involved in radioprotection. Cytokines; IL-8 and IL-IRA, the chemokine, MCP-1, and the growth factor HGF were not significantly different in the CRX-EEMos compared to either control monocytes or EEMos. Other proteins assayed by the kit, such as IL-12 (p40/p70), IL-1b and IFNα/IFNγ were below the limit of detection in all three groups.

TABLE 2
Multiplex Luminex assay of secreted factors by
monocytes and educated monocytes

| Analyte | Monocytes | | EEMos | | CRX-EEMos | |
|---|---|---|---|---|---|---|
| IL-6 | 40 | pg/ml | 60 | pg/ml | 3000 | pg/ml *** $$$ |
| G-CSF | — | | — | | 600 | pg/ml***$$$ |
| IL-10 | 1 | pg/ml | 1 | pg/ml | 25 | pg/ml***$$$ |
| CCL-5 | — | | — | | 50 | pg/ml***$$$ |
| (Rantes) | | | | | | |
| MIP-1a | 300 | pg/ml | 300 | pg/ml | 16000 | pg/ml***$$$ |
| MIP- 1b | 400 | pg/ml | 500 | pg/ml | 12500 | pg/ml***$$$ |
| MCP-1 | 30000 | pg/ml | 30000 | pg/ml | 32000 | pg/ml |
| IL-8 | 17000 | pg/ml | 21000 | pg/ml | 22000 | pg/ml |
| HGF | 30 | pg/ml | 25 | pg/ml | 35 | pg/ml |
| GM-CSF | — | | — | | 137 | pg/ml***$$$ |
| IL-1RA | 1750 | pg/ml | 1750 | pg/ml | 1350 | pg/ml |
| TNF-a | — | | — | | 260 | pg/ml***$$$ |
| IL-13 | 1.5 | pg/ml | 1.5 | pg/ml | 6 | pg/ml***$$$ |
| IL-1b | — | | — | | 11 | pg/ml |
| IL-12 | — | | — | | 1.75 | pg/ml |
| IL-15 | 26 | pg/ml | 24 | pg/ml | 26 | pg/ml |
| EGF | 12 | pg/ml | 15 | pg/ml | 20 | pg/ml |
| IFNa | 2 | pg/ml | 2.7 | pg/ml | 2.8 | pg/ml |

Statistical significance of mean levels of factors secreted by monocytes educated with CRX-EV (CRX-EEMos) compared to control monocytes. *P≤0.05, **P≤0.005, ***P≤0.0005. Statistical significance of secreted levels of CRX-EEMos compared to EEMos. $P≤0.05, $$P≤0.005, $$$P≤0.0005. (−) below limit of assay detection.

Gene expression of educated monocytes assessed by qPCR: An additional gene expression study on several proteins found elevated by secretome analysis was performed on CRX-EEMos and compared to control monocytes and EEMos. Monocytes were exposed ex vivo to CRX-EV to produce CRX-EEMos. Monocytes were separately exposed to MSC-exosomes to produce control EEMos and untreated monocytes served as controls. Gene expression for secreted proteins IL-13, G-CSF, GM-CSF, CCL-5 MIP-1a and MIP-1b, was significantly higher (P≤0.05, P≤0.005) in the CRX-EEMos compared to the control monocytes (Table 3). A subset of these genes G-CSF, MIP-1a and MIP-1b were also higher in the CRX-EEMos than in the EEMos. (P≤0.05). IL-13 and GM-CSF were found to be significantly elevated in the EEMos compared to the controls. Furthermore, important genes involved in radioprotection (G-CSF, GM-CSF) were found to be elevated.

TABLE 3
Gene expression of CRX-EEMOs by qPCR

| | Control | | |
|---|---|---|---|
| Gene | monocytes | EEMos | CRX-EEMos |
| IL-13 | 1.0 | 7.1* | 15** |
| G-CSF | 1.0 | 3.4 | 548**$ |
| GM-CSF | 1.0 | 3.2* | 747* |
| CCL-5 | 1.0 | 1.2 | 6.4* |
| MIP-1a | 1.0 | 1.4 | 45.4**$ |
| MIP-1b | 1.0 | 1.3 | 7.7***$ |

Expression levels of monocytes educated with MSC-exosomes (EEMos) or CRX-exosomes (CRX-EEMos) compared to control monocytes. *P≤0.05, **P≤0.005, ***P≤0.0005. Expression levels of CRX-EEMos compared to EEMos. $P≤0.05, $$P≤0.005, $$$P≤0.0005.

Flow cytometry was used to characterize changes in key monocyte surface markers after education. Surface markers CD86 and HLA-DR are considered pro-inflammatory M1 markers, while CD163 and CD206 are anti-inflammatory M2 markers. CD16 is also considered a pro-inflammatory macrophage/monocyte marker, while PD-L1, PD-L2, and CD73 are all markers for an immunosuppressive cell phenotype. EEMos, LPS-EEMos, CRX-EEMos and CRX-pulse-EEMos showed significant differences in surface marker expression between the groups. (FIG. 3) EEMos were characterized by high expression of CD163, CD16 and CD86 and low CD73. LPS-EEMos and CRX-EEMos were most similar, with both showing very high expression of PD-L1 but low expression of CD16 and CD86. CRX-pulse-EEMos were somewhat like the CRX-EEMos but showed significantly higher levels of CD206 and CD73 and lower levels of PD-L1. Overall, CRX-EEMos displayed a more immunosuppressive and anti-inflammatory phenotype like LPS-EEMos, but very distinct compared to EEMos.

### Example 3: Educating Cells Ex Vivo with CRX-EV in an Immunocompromised ARS Mouse Model

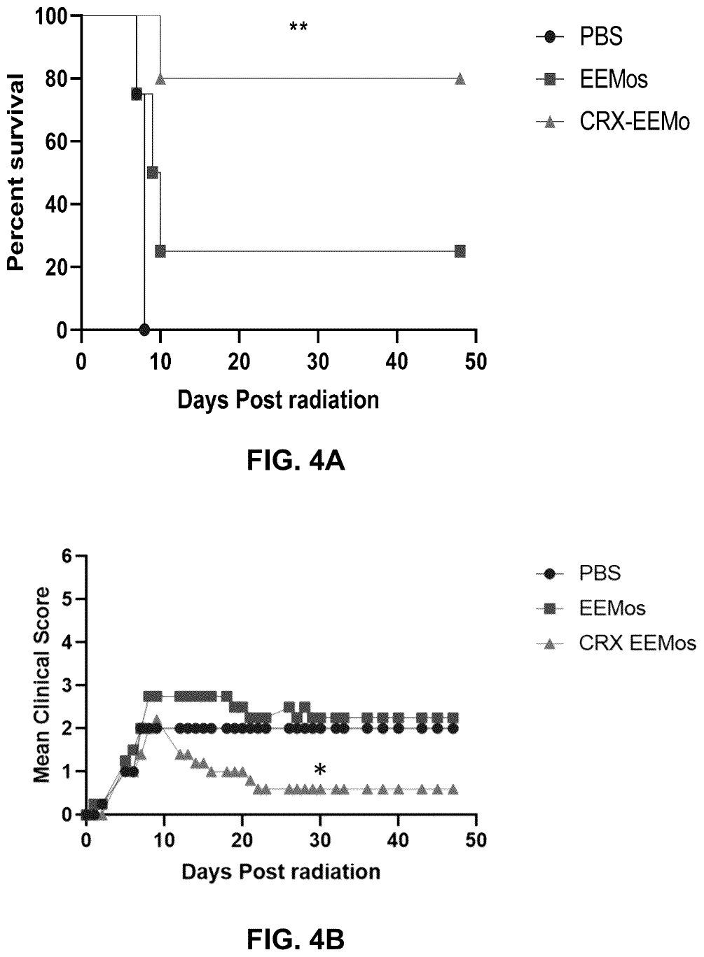

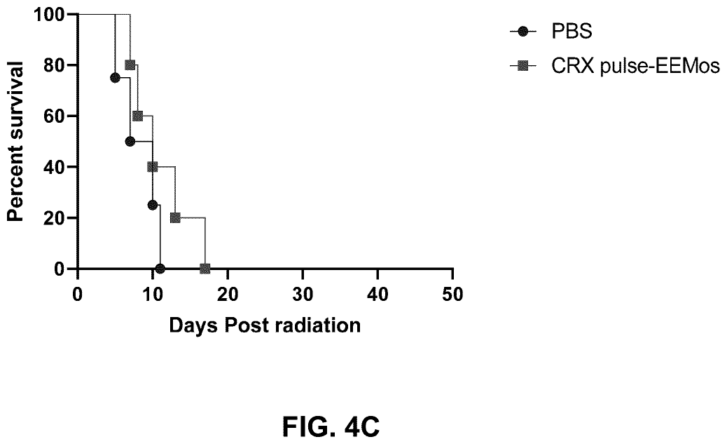

Four hours after a 4 Gy lethal radiation dose, a single dose of 1×10⁷ educated monocytes was administered i.v. in a xenogeneic ARS mouse model. CRX-EEMos were significantly more effective than EEMos or PBS in protecting these mice from the lethal effects of ARS (FIG. 4A). The health of the mice, assessed by clinical scores and % weight loss, was also significantly improved in mice treated with CRX-EEMos (FIG. 4B). In contrast, CRX-pulse-EEMos did not effectively protect the mice from ARS (FIG. 4C). Recovery from ARS was assessed by monitoring CBC in mice before and at 5 days and 29 days after radiation exposure. While the CBC in the test groups declined at day 5, the white blood cell count (WBC) of neutrophils, lymphocytes, and monocytes improved to pre-radiation levels at day 29 in the mice treated with CRX-EEMos. This recovery was not evident in the surviving EEMos-treated mouse (Table 4).

TABLE 4
CRX-EEMos helped restore complete blood cell count
(CBC) of mice in an Immunocompromised ARS model

| | Day post | RBC | WBC | Neutrophils | Lymphocytes | Monocytes | Platelets |
|---|---|---|---|---|---|---|---|
| Group | radiation | (M/μl) | (K/μl) | (K/μl) | (K/μl) | (K/μl) | (K/μl) |
| Pre-rad | n/a | 9.4 | 1.78 | 1.73 | 1.0 | 0.16 | 1124 |
| Control | | | | | | | |
| PBS | 5 | 10.5 | 0.3** | 0.07* | 0.18 | 0.03* | 470** |
| EEMos | 5 | 9.4 | 0.2** | 0.05* | 0.05 | 0.02* | 342** |
| CRX-EEMos | 5 | 8.4 | 0.35** | 0.05* | 0.13 | 0.03* | 260** |
| EEMos | 29 | 4.72 | 0.38* | 0.17 | 0.15 | 0.05 | 247 |
| CRX-EEMos | 29 | 7.5* | 1.4 | 0.85 | 0.33 | 0.18 | 268** |

*P ≤ 0.05,
**P ≤ 0.005

### Example 4: Educating Cells In Vivo with CRX-EV in an Immunocompromised ARS Mouse Model

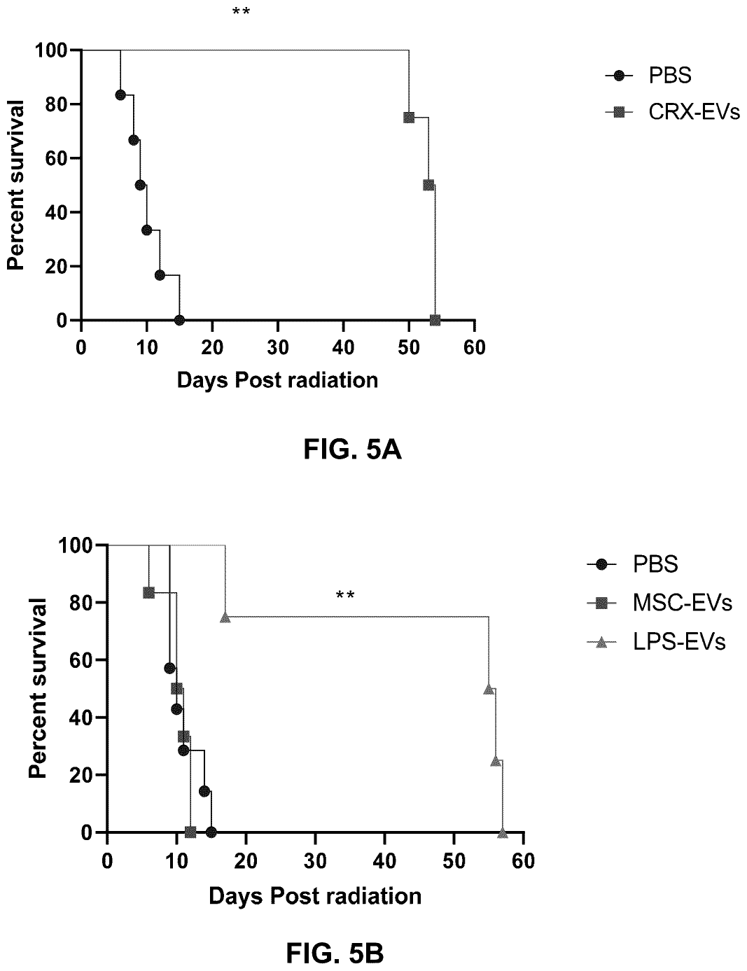

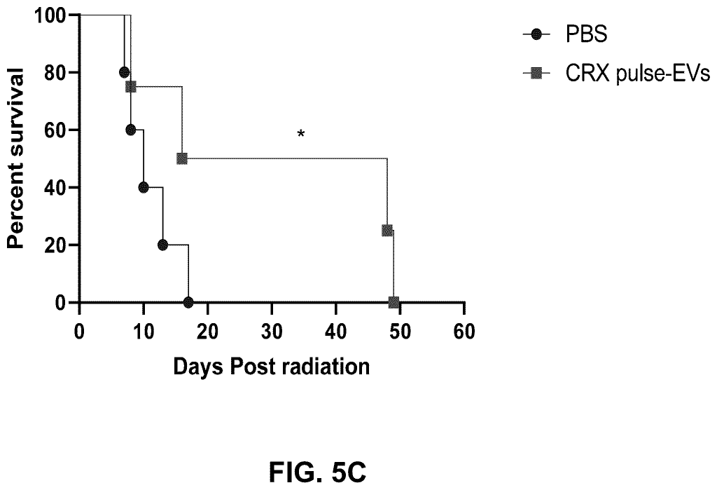

Direct treatment of mice with CRX-EV effectively protected against ARS in the mouse model (using NSG mice). Four hours after a 4 Gy lethal radiation dose, a single i.v. dose of 5×10⁹ CRX-EV administered i.v. in the ARS mouse model significantly protected the mice compared to PBS with 100% survival up to about day 50 (FIG. 5A). CRX-EV also significantly improved the health of these mice compared to controls, as determined by mean clinical score (data not shown). Recovery from ARS was assessed by monitoring CBC in mice at day 6 and day 30 compared to the CBCs in the mice before radiation exposure (Pre-rad Control). While the CBC in the all test groups declined at day 6, at day 30 the WBC of mice treated with CRX-EV improved to pre-radiation levels (Table 3). Treatment with LPS-exosomes protected the mice in the ARS model to a similar extent as the CRX-EV treatment. In contrast, MSC-exosomes did not prevent lethality the ARS model (FIG. 5B). In addition, treatment of the mice with 2-hour CRX-pulse-EVs showed significant protection against ARS (50% survival) compared to PBS, but its effect was not a dramatic as with CRX-EV (FIG. 5C). Comparing the % survival of the mice in the ARS model, the CRX-pulse-EVs were more effective when used for in vivo education of monocytes (FIG. 5C) compared to the ex vivo education of monocytes producing CRX-pulse-EEMos (FIG. 4C). The results indicated that direct treatment of mice with CRX, CRX-EVs, CRX-pulse-EVs, or LPS-EVs was effective in the ARS model. Since the exosomes used for in vivo education were all stored frozen before use, this treatment approach provides for the simplest and least-expensive off-the-shelf in vivo therapy to treat radiation injury. Recovery from ARS after in vivo education of cells with CRX-EV was also assessed by monitoring CBC in mice at various times post-radiation exposure. Pre-radiation CBC values in normal control mice were compared to treated mice at day 6 and day 30. While the CBC in the test groups significantly declined at day 6, the WBC was partially restored in mice treated with CRX-EV that were not significantly different for the normal controls (Table 5).

TABLE 5
CRX-EVs helped to restore CBC of mice in an Immunocompromised ARS model

| | | | | | | | | Platelet |
|---|---|---|---|---|---|---|---|---|
| | Day post | RBC | WBC | Neutrophils | Lymphocytes | Monocytes | Platelets | volume |
| Group | radiation | (M/μl) | (K/μl) | (K/μl) | (K/μl) | (K/μl) | (K/μl) | (K/μl) |
| Normal | n/a | 8.658 | 2.165 | 1.187 | 0.62 | 0.228 | 929.85 | 4.85 |
| Control | | | | | | | | |
| PBS | 6 | 7.03 | 0.11*** | 0.01* | 0.01* | 0.01** | 140*** | 5.07 |
| CRX-EVs | 6 | 7.756 | 0.213** | 0.01* | 0.01* | 0.01* | 118.33*** | 5.86* |
| CRX-EVs | 30 | 6.26 | 0.88 | 0.62 | 0.2 | 0.04* | 372.5* | 6.3* |

*P ≤ 0.05,
**P ≤ 0.005,
***P ≤ 0.0005

### Example 5: Educating Cells In Vivo with CRX-EV in an Immunocompetent ARS Mouse Model

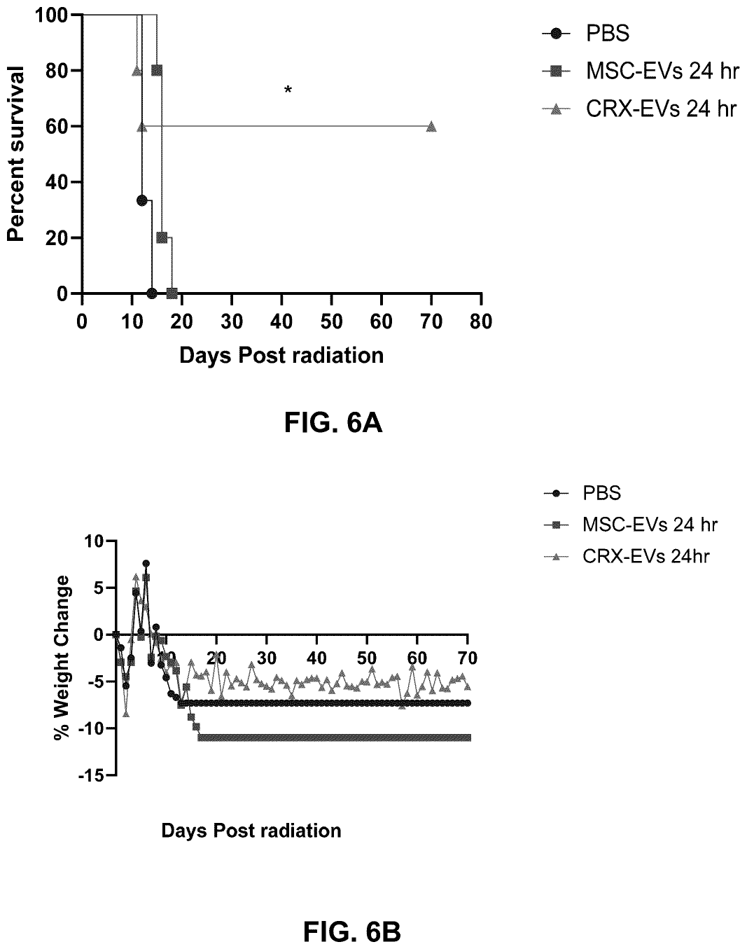

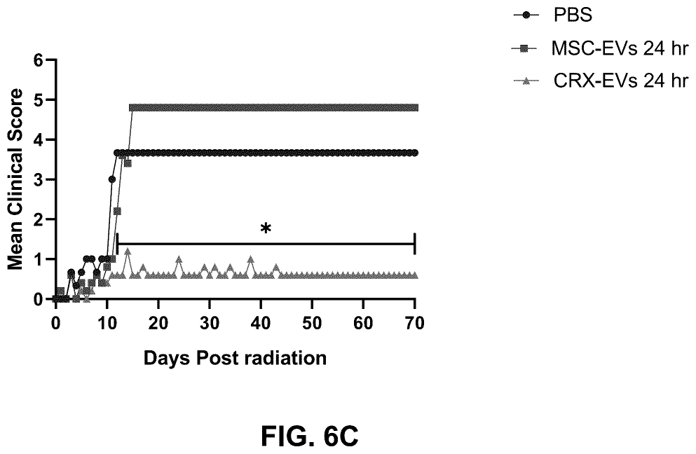

Direct treatment of immunocompetent ICR mice with CRX-EV effectively protected against ARS in this mouse model. Twenty-four hours after a 9 GY lethal radiation dose, a single i.v. dose of 5×10⁹ CRX-EV administered i.v. in the ARS mouse model significantly (P≤0.05) protected the mice long-term compared to PBS or MSC-EV with significant survival (60%) beyond day 50 and up to day 70 (FIG. 6A). The health of the mice, assessed by percent weight change and mean clinical score, was also significantly improved in mice treated with CRX-EV (FIG. 6B, showing percent weight change). CRX-EV also significantly (P≤0.05) improved the health of the mice compared to controls, as determined by mean clinical score (FIG. 6C). These data also illustrate that treatment with CRX-EV can begin 24 hours after radiation exposure and a single dose can provide long-lasting protection beyond 50 days. Based on this long term survival results, the ICR mice treated with CRX-EV completely recovered after lethal radiation exposure are expected to die only from causes unrelated to the radiation exposure.

Recovery from ARS was also determined by monitoring the CBC in surviving mice at day 6, day 20 and 41 compared to the CBCs in the mice before radiation exposure (Pre-rad Control). While the CBC (K/ul) for most blood cell types, (WBC, neutrophils, lymphocytes, monocytes and platelets) significantly declined in the all test groups at day 6, at day 20 the WBCs, lymphocytes, monocytes of surviving mice treated with CRX-EV improved to pre-radiation levels (Table 6).

TABLE 6
CBC recovery evaluation of the immunocompetent ARS model

| | | | | | | | | Platelet |
|---|---|---|---|---|---|---|---|---|
| | Day post | RBC | WBC | Neutrophils | Lymphocytes | Monocytes | Platelets | volume |
| Group | radiation | (M/ul) | (K/ul) | (K/ul) | (K/ul) | (K/ul) | (K/ul) | (fL) |
| Pre-rad | n/a | 7.5 | 8.1 | 1.7 | 5.8 | 0.45 | 852 | 5.1 |
| Control | | | | | | | | |
| PBS | 6 | 8.9 | 0.3*** | 0.05*** | 0.17*** | 0.04** | 203*** | 6.0*** |
| MSC-EVs | 6 | 8.8 | 0.2*** | 0.02*** | 0.06*** | 0.02** | 276** | 6.0*** |
| CRX-EVs | 6 | 7.9 | 0.2*** | 0.02*** | 0.1* | 0.03** | 192*** | 6.3*** |
| CRX-EVs | 20 | 7.9 | 5.6 | 0.7* | 4.5 | 0.26 | 434* | 6.2*** |
| CRX-EVs | 41 | 9.9 | 5.5 | 2.2 | 2.7* | 0.59 | 762 | 5.2 |

*P ≤ .05,
*P ≤ .005,
***P ≤ .0005

In contrast, MSC-EV did not prevent lethality the ARS model and all the mice died in that group by day 18 (FIG. 6A). By day 41, both neutrophils and platelets in the CRX-EV treated mice improved to the normal pre-radiation levels.

Another observation was the difference in baseline CBCs counts in the pre-radiation controls of immunocompetent mice (ICR) vs. baseline CBC counts in the pre-radiation immunocompromised mice (NSG) described in Example 4. Lymphocytes were much higher in the ICR mice. Complete recovery of platelets were seen at Day 41 in ICR mice, which was rarely seen during this period in NSG mice.

Long-term sustained survival and lack of morbidity was observed in the experiments described in this Example. Therefore, the studies described in this example using NSG mice and ICR mice demonstrate that CRX-EVs can be effective in subjects with different degrees of immune competence.

### Example 6: Educating Cells Ex Vivo with CRX without MSC

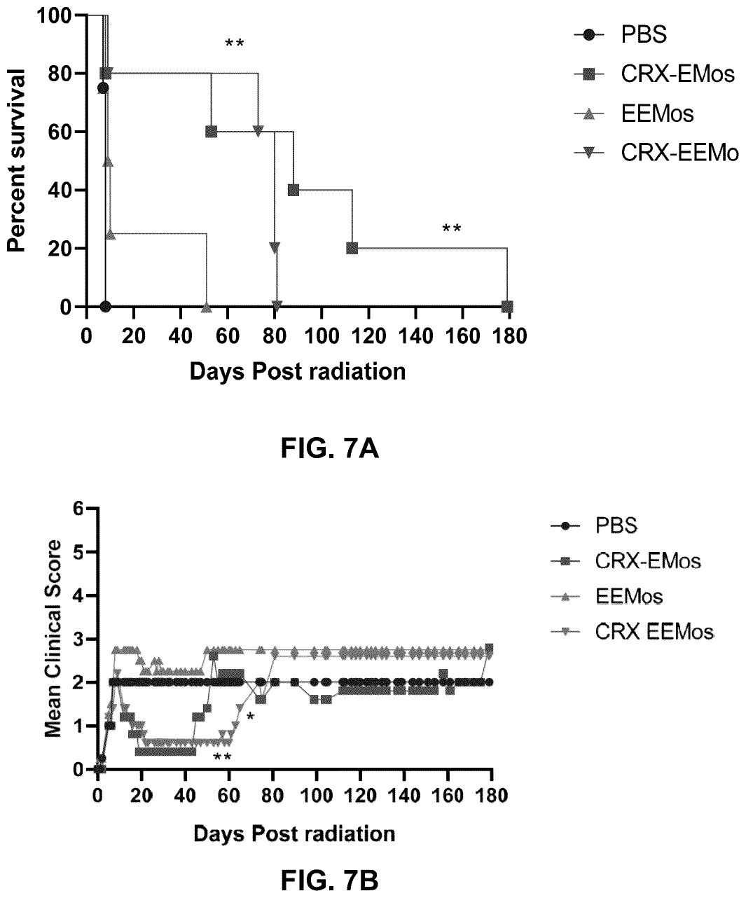

In addition to treating mice in the ARS model using CRX-EV, it was also possible to eliminate the need to use MSC-EV to educate monocytes. CRX can directly stimulate monocytes; specifically, 1×10⁷ monocytes were treated with CRX (100 ng/ml) for 24 hours in 10 mls of complete macrophage/monocyte media, harvested by centrifugation and washed with PBS. The resulting CRX educated monocytes (CRX-EMos) (monocytes directly exposed ex vivo to CRX or CRX-EV) were significantly more effective than EEMos or PBS at preventing lethality in the NSG ARS model with NSG mice. (FIG. 7A). Treatment of mice with CRX EV-educated monocytes (CRX-EEMos) served as a positive (effective) treatment comparator. Efficacy of the CRX-EMos was similar to the CRX exosome-educated monocytes (CRX-EEMos). CRX-EMos prevented lethality to an extent similar to CRX-EEMos in the NGS ARS mouse model, although long-term efficacy started to waned by day 50 (FIG. 7A). The clinical scores of mice after treatment with CRX-EMos was also like those observed following treatment with CRX-EEMos, although protection waned in the former at about day 42 (FIG. 7B). Treatment with CRX-EMos also improved CBC recovery after radiation exposure. While CBC in treated mice declined in all test groups at day 5, WBC improved in CRX-EMos-treated mice at day 29 to pre-radiation levels. (Table 7). When compared to the results observed after treatment with CRX-EEMos (the positive treatment comparator), after treatment with CRX-EMos, CBC levels were as good for WBC groups (neutrophils, lymphocytes) and even better recovery of platelet levels was observed at day 29. These results indicate that while CRX-EEMos can provide long-term protection in the ARS model, the simplicity of using direct CRX monocyte treatment makes it an alternative therapeutic approach.

TABLE 7
CRX-EMos helped restore CBC of mice in an ARS model

| | | | | | | | | Platelet |
|---|---|---|---|---|---|---|---|---|
| | Day post | RBC | WBC | Neutrophils | Lymphocytes | Monocytes | Platelets | volume |
| Group | radiation | (M/μl) | (K/μl) | (K/μl) | (K/μl) | (K/μl) | (K/μl) | (K/μl) |
| Pre-rad | n/a | 9.4 | 1.78 | 1.73 | 1.0 | 0.16 | 1124 | 4.9 |
| Control | | | | | | | | |
| PBS | 5 | 10.5 | 0.3** | 0.07* | 0.18* | 0.03* | 470** | 4.5* |
| CRX-EMos | 5 | 10.7 | 0.3** | 0.1* | 0.27* | 0.03* | 388** | 4.9 |
| EEMos | 5 | 9.4 | 0.2** | 0.05* | 0.05* | 0.02* | 342** | 4.9 |
| CRX EEMos | 5 | 8.4 | 0.35** | 0.05* | 0.13* | 0.03* | 260** | 5.1 |
| CRX-EMos | 29 | 7.6 | 2.4 | 1.2 | 1.0 | 0.06 | 727 | 5.3 |
| EEMos | 29 | 4.72 | 0.38 | 0.17 | 0.15 | 0.05 | 247 | 6.8 |
| CRX-EEMos | 29 | 7.5* | 1.4 | 0.85 | 0.33 | 0.18 | 268** | 5.9** |

*P ≤ 0.05,
**P ≤ 0.005

The foregoing demonstrates several therapeutic modalities for effectively treating ARS using a synthetic TLR4 agonist. Stimulation of human MSCs with CRX produced CRX-EVs having elevated surface marker expression of CD44, CD29, and CD146. Monocytes educated ex vivo using CRX-EVs (CRX-EEMos) effectively treated ARS in the mouse model. The CRX-EEMos showed increased IL-6, IDO, FGF2, IL-10, IL-12, IL-7, IL-8, IL1B, VEGFA, NFKB, and IL-23 gene expression. By flow cytometry, the CRX-EEMos showed a surface marker profile characterized by high levels of PD-L1 but low levels of CD16 and CD86. The CRX-EEMos helped repair radiation-induced bone marrow injury by restoring the CBC of mice in the ARS model. Direct treatment of mice with CRX-EVs was effective in the ARS mouse model. CRX-EVs were capable of educating monocytes and macrophages in situ, as monocytes and macrophages were less likely to be affected by radiation damage than other cell types. The use of CRX alone for the direct stimulation of monocytes ex vivo was also effective in the ARS mouse model. These results also indicate the potential for a fourth approach, the direct treatment of mice with CRX-527 to be effective in the ARS model.

### Example 7: Educating Cells In Vivo with CRX-EV in a GVHD Model

To determine whether the in vivo treatment of extracellular vesicles (EVs) from CRX-stimulated MSCs could suppress human T-cell activation leading to graft vs. host disease (GVHD), mice were administered EVs from CRX-stimulated MSCs (CRX-EV) at various times post-transplant.

Immunodeficient NSG mice were transplanted with human peripheral blood mononuclear cells (PB-MNC) or isolated human T cells (PB-Tc). Mice were monitored for overall survival and the number of T cells in the liver (a GVHD target organ) upon euthanasia or the experiment endpoint.

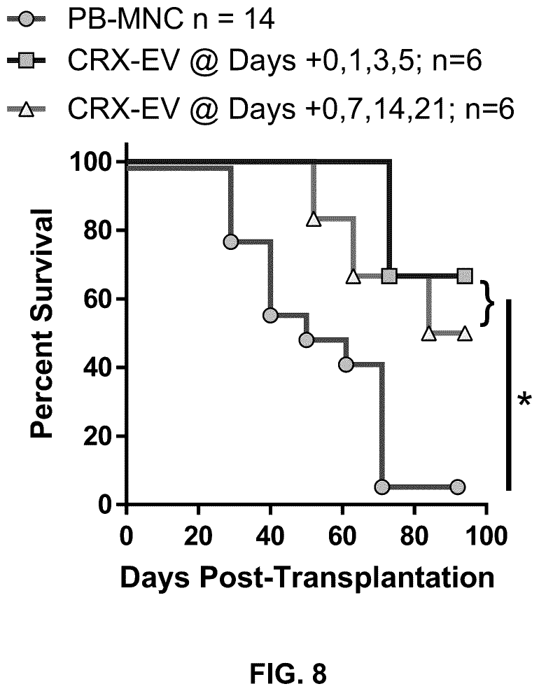

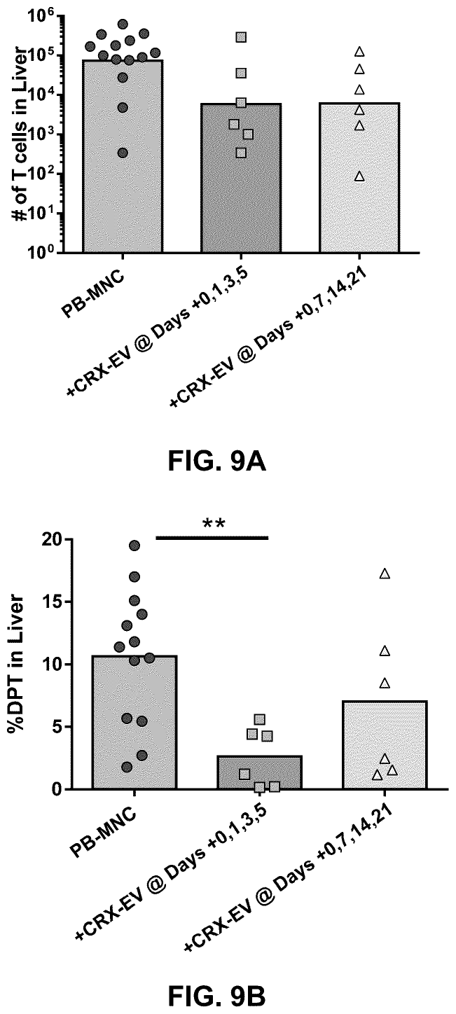

Early (days +0, 1, 3, 5) or late (days +0, 7, 14, 21) post-transplant treatment of EVs from CRX stimulated MSCs (CRX-EV) increased overall survival of mice compared to untreated PB-MNC control. (FIG. 8). The CRX-EVs administered on day zero were administered concurrently with the PB-MNC transplant. Livers from mice in FIG. 8 were isolated, processed and quantified for overall number of human T cells (FIG. 9A) and the percentage of a highly pathogenic CD4⁺/CD8⁺ double positive T cells (DPT) population (FIG. 9B). While the overall number of T cells in the liver did not differ between treatments, early treatment of CRX-EV post-transplant was able to significantly reduce the number of pathogenic DPT in the livers of these mice.

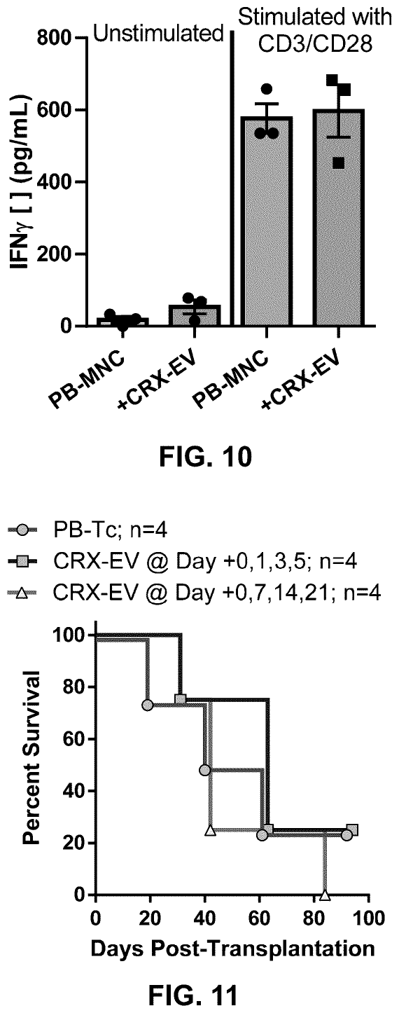

The mechanism of CRX-EV mediated GVHD suppression was also investigated. EVs from CRX-stimulated-MSCs were exposed to human T cells in vitro that were either unstimulated or stimulated with CD3 and CD28 antibodies. The results demonstrated that CRX-EVs do not have any direct suppressive activity on human T cells (FIG. 10). These results further suggested that the target of the CRX-EV is a myeloid cell population.

To further confirm that CRX-EV have no direct suppressive effect on human T cells, isolated human T cells (PB-Tc) were transplanted into NSG mice followed by the in vivo treatment of CRX-EVs. No difference was detected in the mice treated with CRX-Ev versus untreated controls (FIG. 11). This further supported the conclusion that CRX-Ev did not have any direct effect on human T cells and require human monocytes to successfully suppress GVHD.

In mice that survived after the treatment of CRX-EV, the level of IFNγ in the serum was significantly lower compared to mice that died. This suggested that T cell activation is actively suppressed in the mice that survive (data not shown). Additionally, the number of T cells between mice that survived and died was not significantly different highlighting the selective inhibition of T cell activation, not T cell survival, as a possible mechanism of CRX-EV suppression of GHVD.

Thus, both treatment regimens (early interval administration of CRX-EV and late interval administration of CRX-EV) showed a suppressive effect against GVHD. Observational and GVHD scoring suggests that early treatment can have a greater suppressive effect. Furthermore, continuous administration of CRX-EV at, for example, 1, 3, 5, 7, 14, and 21 days, can further improve GVHD suppression and overall survival.

The results described in this Example in the GVHD model and the results described above in Examples 1-6 with respect to the ARS model indicate that CRX-EVs, either used to educate monocytes and macrophages ex vivo or in vivo, have the potential to help recovery after whole-body radiation and prevent excessive donor T cell activation leading to GVHD after a bone marrow transplantation.

### Example 8: Educating Cells In Vivo with CRX-EV in Acute Kidney Injury Models

To determine if CRX-EVs can improve survival in an acute kidney injury (AKI) mouse model, mice experiencing AKI were administered CRX-EVs.

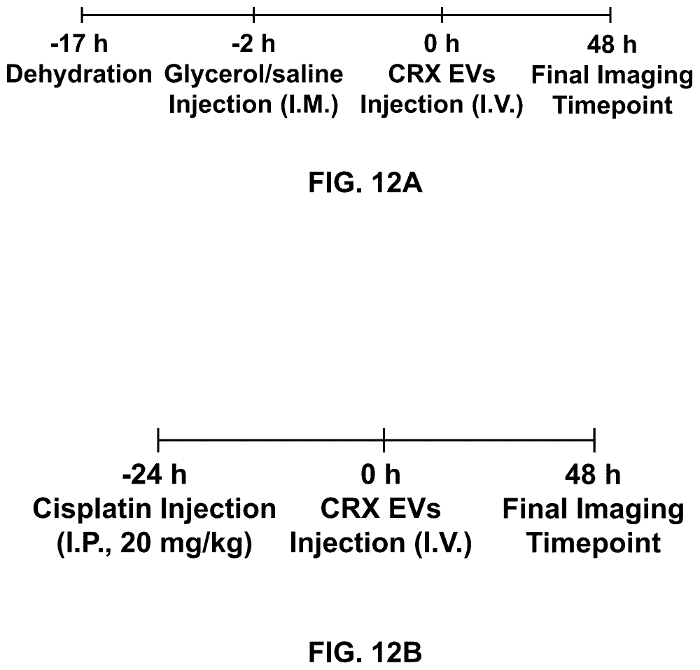

AKI Models: Two AKI models were established: rhabdomyolysis-induced AKI (RM-AKI) and cisplatin-induced AKI (CP-AKI). To establish the RM-AKI model, mice were dehydrated for 17 hours prior to being administered CRX-EVs. At two hours prior to administration of CRX-EVs, the mice were given an intramuscular (IM.) injection of glycerol/saline. CRX-EVs were then administered to the mice by intravenous (IV.) injection (FIG. 12A). To establish the CP-AKI model, mice were administered a large dose (20 mg/kg) of cisplatin by intraperitoneal injection (IP.) 24 hours prior to administering CRX-EVs via I.V. injection (FIG. 12B).

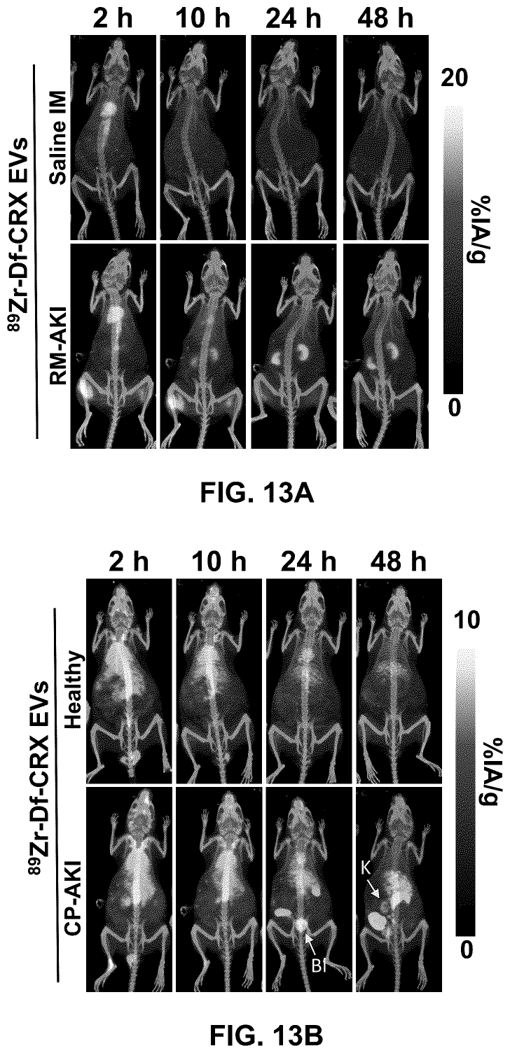

PET Imaging: For imaging, EVs were conjugated with a chelator and radiolabeled (⁸⁹Zr-Df). Images were taken at 2 hours, 10 hours, 24 hours, and 48 hours after I.V. injection with ⁸⁹Zr-CRX-EVs in both the RM-AKI model and the CP-AKI model. PET imaging in the AKI models showed dramatic change in CRX-EVs biodistribution to target injured kidneys. Following RM-AKI model induction, significant CRX-EVs uptake was observed in the injured muscles and kidneys as early as 2 hours and 10 hours post-injection, respectively, compared to mice given saline intramuscularly or healthy mice (FIG. 13A and FIG. 13B). A significant reduction in kidney signal was observed with monocytes and macrophages were depleted using chlodronate in injured mice, which indicated the targeting of monocytes/macrophages by CRX-EVs in vivo. In the CP-AKI model, renal uptake in the injured kidneys was observed after 48 hours post-injection (indicated by the K in FIG. 13B) and in the bladder (indicated by the Bl in FIG. 13B) after 24 hours post-injection.

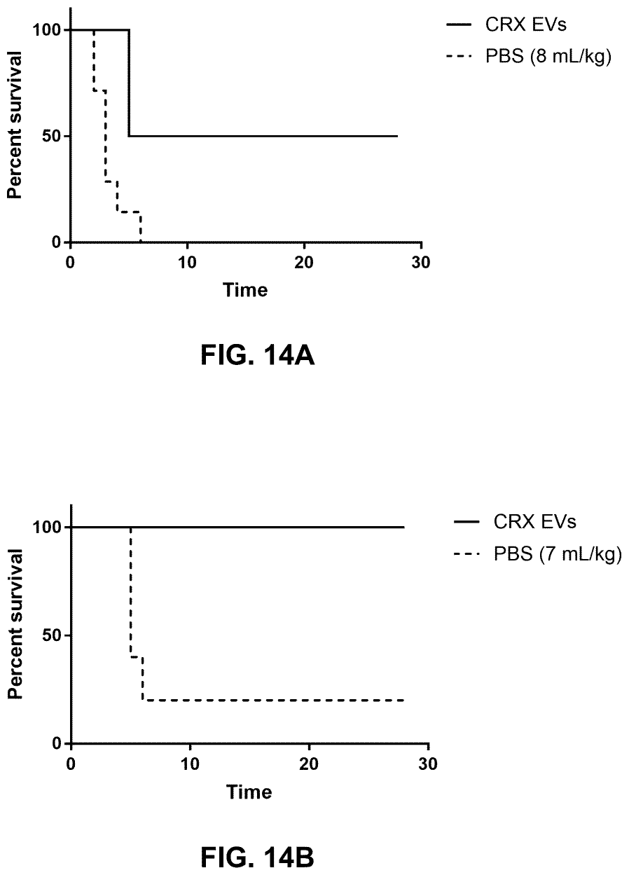

Survival studies: A survival study was performed using the RM-AKI mouse model. Two doses of glycerol were used to induce AKI: 8 mL/kg and 7 mL/kg. Mice (n=4 for the 8 mL/kg model and n=3 for 7 mL/kg group) were then administered 1×10⁹ CRX-EVs by I.V. injection. Injection with PBS was used as a control in both models (n=5). In the 8 mL/kg glycerol group, administering CRX-EVs resulted in 50% survival after 5 days (FIG. 14A). In the 7 mL/kg glycerol group, administering CRX-EVs resulted in 100% survival.

In summary, the Examples described herein demonstrate a number of different approaches to using lipid A AGP molecules (e.g., CRX molecules) to treat and/or prevent various inflammatory diseases or injuries, including acute radiation syndrome, acute radiation injury, GVHD, and acute kidney injury: (1) ex vivo education of cells with CRX-EVs (CRX-EEMos); (2) ex vivo education of cells with CRX (CRX educated monocytes, CRX-EMos); and (3) in vivo education of cells with CRX-EVs. These different approaches will allow for flexible treatment modalities depending on the clinical circumstances.

While some embodiments have been illustrated and described in detail in the appended drawings and the foregoing description, such illustration and description are to be considered illustrative and not restrictive. Other variations to the disclosed embodiments can be understood and effected in practicing the claims, from a study of the drawings the disclosure, and the appended claims. The mere fact that certain measures or features are recited in mutually different dependent claims does not indicate that the combination of these measures or features cannot be used. Any reference signs in the claims should not be construed as limiting the scope.

We claim:

1. A method for generating an educated CD14⁺ cell, the method comprising:
co-culturing a CD14⁺ cell in vitro (i) with an extracellular vesicle isolated from a mesenchymal stromal cell previously exposed to a synthetic lipid A aminoalkyl glucosaminide phosphate (AGP) molecule or (ii) with a synthetic lipid A AGP molecule, until the CD14⁺ cell acquires an anti-inflammatory macrophage or monocyte phenotype.

2. The method of claim 1, wherein the CD14⁺ cell is co-cultured with the extracellular vesicle or the synthetic lipid A AGP molecule for at least 2 hours.

3. The method of claim 1, wherein the mesenchymal stromal cell was exposed to the synthetic lipid A AGP molecule for at least 2 hours prior to isolating the extracellular vesicle.

4. The method of claim 1, wherein the synthetic lipid A AGP molecule is a lipid A mimetic in which the reducing sugar of lipid A has been replaced with an N-acylated aminoalkyl aglycon unit containing an 1-serine-based aglycon unit and three (R)-3-n-alkanoyloxytetradecanoyl residues comprised of varied even-numbered normal fatty acyl chains between 6 and 14 carbon atoms in length.

5. The method of claim 4, wherein the synthetic lipid A AGP molecule is a CRX molecule.

6. The method of claim 5, wherein the CRX molecule comprises three secondary acyl chains of different lengths.

7. The method of claim 5, wherein the CRX molecule comprises three secondary acyl chains each having 10 carbons in length.

8. The method of claim 5, wherein the CRX molecule is CRX-527.

9. The method of claim 3, wherein the mesenchymal stromal cell is exposed to about 20 ng/ml to about 2000 ng/ml of CRX.

10. The method of claim 1, wherein the extracellular vesicle has detectable cell surface markers for CD44, CD29, CD146, CD63, CD81, CD9, and SSEA-4

11. The method of claim 5, wherein the CD14+ cell is co-cultured with about 20 ng/ml to about 2000 ng/ml of the CRX molecule.

12. The method of claim 1, wherein the CD14⁺ cell is a macrophage or a monocyte.

13. A population of educated CD14⁺ anti-inflammatory monocytes or macrophages produced by the method of claim 1, wherein the anti-inflammatory monocyte or macrophage phenotype is characterized by high expression levels of cell surface markers HLA-DR, PD-L1, CD73, and secreted proteins IL-13, G-CSF, GM-CSF, CCL-5, MIP-1a, and MIP-1b and low expression levels of cell surface markers CD16, and CD86, as compared to control CD14⁺ cells.

14. A method for treating or preventing an inflammatory disease or injury in a subject in need thereof, the method comprising:
administering to the subject a therapeutically effective amount of the educated CD14+ cell population of claim 13.

15. The method of claim 14, wherein between about 1×10⁵ cells/kilogram and about 10×10⁹ cells/kilogram of body weight is administered to the subject.

16. The method of claim 14, wherein the inflammatory disease or injury is acute radiation injury, acute radiation syndrome, acute kidney injury, graft-versus-host-disease, or Coronavirus Disease 2019 (COVID-19).

17. The method of claim 16, wherein the ARS is from ionizing radiation.

18. The method of claim 16, wherein the acute radiation injury is from a whole body radiation procedure.

19. The method of claim 18, wherein the whole body radiation procedure occurs before or after a bone marrow transplant.

20. A method for generating an extracellular vesicle, the method comprising:
co-culturing a mesenchymal stromal cell with a CRX molecule for at least 2 hours, wherein an extracellular vesicle is formed, and
optionally isolating extracellular vesicles from the co-culture, wherein an extracellular vesicle is formed.

21. The method of claim 20, wherein the CRX molecule is CRX-527.

22. The method of claim 20, wherein the mesenchymal stromal cell is co-cultured with about 20 ng/ml to about 2000 ng/ml CRX.

23. A population of extracellular vesicles produced by the method of claim 20, wherein the extracellular vesicles are characterized by high expression levels of CD44, CD29, and CD146 as compared to a control extracellular vesicle isolated from a mesenchymal stromal cell not exposed to CRX.

24. The population of extracellular vesicles of claim 23, wherein the EVs comprise exosomes having a diameter of an average or mean between about 50 nm and about 200 nm.

25. The population of extracellular vesicles of claim 23, wherein the EVs comprise microvesicles having a diameter of an average or mean between about 300 nm to about 1100 nm.

26. A method for treating an inflammatory disease or injury in a subject in need thereof, the method comprising:
administering to the subject a therapeutically effective amount of the extracellular vesicle population of claim 23.

27. The method of claim 26, wherein the inflammatory disease or injury is acute radiation injury, acute radiation syndrome, acute kidney injury, graft-versus-host-disease, Coronavirus Disease 2019 (COVID-19), or an inflammatory disease.

28. A method for treating acute radiation syndrome in a subject in need thereof, the method comprising:
administering to the subject a therapeutically effective amount of
(i) a CD14⁺ cell that has been co-cultured with an extracellular vesicle isolated from a mesenchymal stromal cell previously exposed to a CRX molecule, generating CRX-EEM or CRX-EEMos;
(ii) an extracellular vesicle isolated from a mesenchymal stromal cell previously exposed to a CRX molecule, generating CRX-EVs; or
(iii) a CD14+ cell treated with a CRX molecule (CRX-EM or CRX-EMos),
wherein the acute radiation syndrome is effectively treated in the subject.

29. The method of claim 28, wherein the subject has been exposed to a high dose of ionizing radiation.

30. The method of claim 28, wherein effective treatment is indicated by an increase in cell types when performing the complete blood count (CBCs) of the subject as compared the CBCs of the subject in the days following an exposure to radiation.

31. The method of claim 30, wherein the cell types comprise white blood cells.

32. The method of claim 28, wherein the subject is administered a single dose of the CD14⁺ cell of step (i) or the extracellular vesicle of step (ii).

33. The method of claim 28, wherein effective treatment is indicated by the subject having reduced symptoms of acute radiation syndrome.

34. The method of claim 28, wherein the subject is immune-competent.

35. A method for treating or preventing graft-versus-host-disease in a subject undergoing a transplant, the method comprising: administering to the subject a therapeutically effective amount
(i) a CD14+ cell that has been co-cultured with an extracellular vesicle isolated from a mesenchymal stromal cell previously exposed to a CRX molecule, generating CRX-EEM or CRX-EEMos;
(ii) an extracellular vesicle isolated from a mesenchymal stromal cell previously exposed to a CRX molecule, generating CRX-EVs; or
(iii) a CD14+ cell treated with a CRX molecule (CRX-EM or CRX-EMos), wherein the graft-versus-host-disease is suppressed in the subject.

36. The method of claim 35, wherein the extracellular vesicle is administered to the subject in multiple doses and in an interval of about every two days to about every seven days.

37. The method of claim 35, wherein the multiple doses comprises administering a first dose of the extracellular vesicle to the subject on the same day as the transplant and a second dose on the first day after the transplant or one week after the transplant.

38. The method of claim 35, wherein the transplant is a bone marrow transplant.

39. A method for treating or preventing acute kidney injury in a subject, the method comprising: administering to the subject a therapeutically effective amount of
(i) a CD14+ cell that has been co-cultured with an extracellular vesicle isolated from a mesenchymal stromal cell previously exposed to a CRX molecule, generating CRX-EEM or CRX-EEMos;
(ii) an extracellular vesicle isolated from a mesenchymal stromal cell previously exposed to a CRX molecule, generating CRX-EVs; or
(iii) a CD14+ cell treated with a CRX molecule (CRX-EM or CRX-EMos), wherein the acute kidney injury is effectively treated in the subject.

40. The method of claim 39, wherein the acute kidney injury is caused by rhabdomyolysis or chemotherapy.

41. The method of claim 39, wherein the extracellular vesicle is administered to the subject prior to or concurrently with the chemotherapy.
